SEQUENCE LISTING (46 sequences)

SEQ ID NO: 1 - DNA, 2112 bp, Mus musculus
  source 1..2112: mol_type = other DNA
atggatgcttctcctgagccccaacagaagggtgggacactggtactggtccgacggcagcctcctgtgtcccagggcttgctggaaacactgaaggccaggctgaagaagagctgcacctgcagtatgccatgtgctcaggctctggtgcagggtctgtttcctgccatacactggctgccccagtaccgccttaaggaatacctagcaggtgatgtcatgtcaggattggtcattggcattatcctggtgccacaggccatagcctactcactgctggctgggttacaacccatctatagcctctacacttccttctttgccaacctgatctacttcctcatgggcacctcccgccatgttaatgtgggcatcttcagcctgctgtgtctcatggtgggtcaggtggtggacagagaactccagttggctggctttgacccttcccaggattctctagggcccaagaacaatgacagcaccctcaacaactcagctacaacactgatcattgggctacaggactgcagacgggactgctatgccattcgggttgccactgcccttactctgatggccgggctttatcaggtcctcatgggtatcctccggctgggcttcgtgtctacctatctctcacaaccactgctcgatggctttgctatgggagcttctgtgaccatcttgacttctcaggcaaaacacatgctgggtgtgcagatccctcggcaccagggcctaggcatggtggtccacacatggttgagcttgctgcagaatgtgggacaggccaacatatgtgatgtggtcaccagtgccctgtgcctaggagtgctgctggcagctaaggaactctcagatcgctaccgacaccgtctgaaggtgccaatacccacagagttatttgttattgtggtggccacaattgtgtcccattttggacagctccatacacggtttgactcgagggtggctggcaacattcccactggttttgtggccccacaggtaccagaccctaagataatgtggcgtgtggccctggatgccatgtccctggccctcgtgggctcagccttctccatctccttggcagaaatgtttgcccgtagtcatggctactctgtcagtgccaaccaagagctgctagctgtgggctgctgcaatgtgctgcctgccttcttccactgttttgccactagtgctgctctgtccaaaactctggtgaagatagccactggctgccagacccagttgtccagtgtggtcagtgccgctgtggtgttgctggtgctgctggtgctggctccactgtttcatgatctgcagcgatgtgtgctagcttgtatcattgttgtcagcctgcggggggcgctgcgcaaggtgaaggatctcccacaactttggcggctaagccctgccgatgcactggtctgggtggctactgcagctacctgtgtgctagtcagcaccgaggctggactattagctggagtgttcttctccctgcttagcctggcaggccgtacacagcgtccacgggctgcccttcttgcccgaattggagactccaccttctatgaggatgctgctgaatttgagggcctcctgcccccacctgaggtgcgagtgttccgtttcacaggtccactttactacgccaacaaggacttcttccttcggtctctctacagactcacagggttggatgcagggcactcagccaccaggaaggatcagggcccggaggtgggtgtcagtaacagaagtcttgttgatggcaaggatctgggttcagtgagcagtggggctgggctggtggtacccctggcatttggtttccacacagtggtcattgactgtgcaccactgctgttcctggatgtggctggcatggccacattgaaagacctccgcagagactacagggccttggacatcaccctgcttctggcttgctgcagtccctcggtgagagacacgttgagaaaagggggcttccttggggaagaacagggagctgaaaatgagctgctattccccagtgtgcacagtgctgtggaggctgcatgtgcccgccgtgaggagctgctggctgctgactctgccctc

SEQ ID NO: 2 - protein, 704 aa, Mus musculus
MDASPEPQQKGGTLVLVRRQPPVSQGLLETLKARLKKSCTCSMPCAQALVQGLFPAIHWLPQYRLKEYLAGDVMSGLVIGIILVPQAIAYSLLAGLQPIYSLYTSFFANLIYFLMGTSRHVNVGIFSLLCLMVGQVVDRELQLAGFDPSQDSLGPKNNDSTLNNSATTLIIGLQDCRRDCYAIRVATALTLMAGLYQVLMGILRLGFVSTYLSQPLLDGFAMGASVTILTSQAKHMLGVQIPRHQGLGMVVHTWLSLLQNVGQANICDVVTSALCLGVLLAAKELSDRYRHRLKVPIPTELFVIVVATIVSHFGQLHTRFDSRVAGNIPTGFVAPQVPDPKIMWRVALDAMSLALVGSAFSISLAEMFARSHGYSVSANQELLAVGCCNVLPAFFHCFATSAALSKTLVKIATGCQTQLSSVVSAAVVLLVLLVLAPLFHDLQRCVLACIIVVSLRGALRKVKDLPQLWRLSPADALVWVATAATCVLVSTEAGLLAGVFFSLLSLAGRTQRPRAALLARIGDSTFYEDAAEFEGLLPPPEVRVFRFTGPLYYANKDFFLRSLYRLTGLDAGHSATRKDQGPEVGVSNRSLVDGKDLGSVSSGAGLVVPLAFGFHTVVIDCAPLLFLDVAGMATLKDLRRDYRALDITLLLACCSPSVRDTLRKGGFLGEEQGAENELLFPSVHSAVEAACARREELLAADSAL

SEQ ID NO: 3 - DNA, 2217 bp, Mus musculus
  source 1..2217: mol_type = other DNA
atgtcttcagaaaataaagagcagcatgacctctcaccgagggacttacctgaagaagcctttggtttcccatctgagctccccctggaggctcaaagaaggtcaggcactgacttgaggcagtctgagaccggtcatggccggagagctttccgtaggatccacatggagctccgtgagaaaccagacacagacatcaaacagtttgtcatcagagagctccagaagagttgccagtgcagcgcagccaaagtcagagacggggcttttgatttctttcctgttttgcggtggctcccaaaatatgacctgaagaaaaacattttaggtgatgtgatgtctggcctgattgtgggtatcctgttggtgccccagtccattgcttactccctgctggctggccaggagcctatatatggtctatatacatcattttttgccagcattatttatttcctgtttggtacatcccgccacatctctgtgggcatttttggaatactgtgccttatgattggtgaggttgttgaccgagaactacataaagcctgccctgacactgatgctacgtcatcttcgatagcagtgttttcaagtggatgtgtggttgtaaaccatacattagacggactctgtgataaaagctgctatgcaattaaaattggcagcactgtgaccttcatggctggagtttatcaggtagcaatgggcttctttcaagtgggctttgtctctgtctacctctccgacgccttgctgagcgggtttgtcactggcgcctccttcaccatcctcacgtctcaggccaagtacctccttgggctgagccttcctcggagtcatggtgtcggctcagtcattactacctggatccacatcttcagaaacatccgtaacaccaacatctgtgatctcatcaccagccttttatgtcttctggtcctcgtgccatccaaagaactcaatgagcacttcaaggacaagctcaaggctccaattccggtcgagctcattgtcgttgtggcggccacattggcttctcattttggaaaactaaatgggaattacaattccagtattgctggacacattcccactgggtttatgccacccaaagcaccagactggagcctcattcctaatgtggctgtagatgcaatagctatttctatcattggttttgctatcactgtatcactttctgagatgtttgccaagaaacatggctacacggtcaaagcaaaccaagaaatgtatgccattggcttttgcaatatcataccttccttcttccactgcataactactagtgcagctcttgcaaagacattggttaaagagtcaacaggctgccagacgcagctgtcagctatagtgacagcccttgtccttttgttggtccttctggtaatagctcctttattctattcccttcaaaaatgtgtccttggtgtgatcactattgtaaatctccggggcgcacttctgaaatttagagacctgccaaagatgtggaggctaagcagaatggatacagttatctggtttgtgactatgctgtcctccgctctgttaagcactgaaataggcctgctggttggggtttgtttttcaatgttctgtgtgatcctccgtactcagaagccaaagaattcactgcttggtttagaagaagaatctgaaacctttgaatccatttccacttacaagaaccttcggagcaagtccggcatcaaggttttccgcttcatagcccctctctactacataaacaaagagtgctttaaatcagctttgtacaagaaagctcttaacccagtcttggtaaaggcagcttggaaaaaggcagcaaagagaaaactcaaagaagaaatggtgacctttcgtggggacccggatgaagtttcaatgcagctttcccatgatcccttggaggtgcacactatcgtgatcgactgtagtgccatacagtttttagacacagcaggaatccacacactgaaagaagttcgccgggattatgaagccgttgggatccaggttttactggcgcagtgcaatccttctgtgagggattccttggccagaggagaatattgcaaaaaggaagaggaaacccttctcttctacagtctgtctgaagcagtagcctttgcagaagactctcagaatcagaaaggggtgtgtgttgtcaacggtctgagtctctctggtgac

SEQ ID NO: 4 - protein, 739 aa, Mus musculus
MSSENKEQHDLSPRDLPEEAFGFPSELPLEAQRRSGTDLRQSETGHGRRAFRRIHMELREKPDTDIKQFVIRELQKSCQCSAAKVRDGAFDFFPVLRWLPKYDLKKNILGDVMSGLIVGILLVPQSIAYSLLAGQEPIYGLYTSFFASIIYFLFGTSRHISVGIFGILCLMIGEVVDRELHKACPDTDATSSSIAVFSSGCVVVNHTLDGLCDKSCYAIKIGSTVTFMAGVYQVAMGFFQVGFVSVYLSDALLSGFVTGASFTILTSQAKYLLGLSLPRSHGVGSVITTWIHIFRNIRNTNICDLITSLLCLLVLVPSKELNEHFKDKLKAPIPVELIVVVAATLASHFGKLNGNYNSSIAGHIPTGFMPPKAPDWSLIPNVAVDAIAISIIGFAITVSLSEMFAKKHGYTVKANQEMYAIGFCNIIPSFFHCITTSAALAKTLVKESTGCQTQLSAIVTALVLLLVLLVIAPLFYSLQKCVLGVITIVNLRGALLKFRDLPKMWRLSRMDTVIWFVTMLSSALLSTEIGLLVGVCFSMFCVILRTQKPKNSLLGLEEESETFESISTYKNLRSKSGIKVFRFIAPLYYINKECFKSALYKKALNPVLVKAAWKKAAKRKLKEEMVTFRGDPDEVSMQLSHDPLEVHTIVIDCSAIQFLDTAGIHTLKEVRRDYEAVGIQVLLAQCNPSVRDSLARGEYCKKEEETLLFYSLSEAVAFAEDSQNQKGVCVVNGLSLSGD

SEQ ID NO: 5 - DNA, 1782 bp, Mus musculus
  source 1..1782: mol_type = other DNA
atgaagctcctcaattatgctttggtctatcgccgctttctccttgtggttttcactattttggttttcttgccactccctctcatcatccgcaccaaggaagcacaatgtgcctacatcctctttgttattgccatattttggatcacagaagccttgcccctgtcaatcacagctctactgcctgggttaatgttccccatgtttgggatcatgcgttcttcacaggtggcttctgcttatttcaaagactttcaccttctgctaattggagtcatctgtttagcaacatcaatagaaaaatggaatttgcacaagaggatcgctctgaggatggtgatgatggtgggggtgaatccagcctggctgacgttggggttcatgagcagcactgccttcttatctatgtggcttagcaacacctctactgctgccatggtgatgcccatcgtggaggctgtggcgcagcagatcatcagtgctgaagcagaggccgaggccactcagatgacttatttcaatgaatctgccgcccacggactggacattgatgaaactgttattggacaagaaacaaatgagaaaaaagagaaaacaaaaccagctccaggaagcagtcatgacaaagggaaagtgtcaagaaagatggagacagaaaagaatgcagtcacaggagcaaaatatcggtcaaggaaggaccacatgatgtgtaagctcatgtgtttatctgttgcttactcttcaaccattggtgggctgacaacaatcacgggtacctccaccaacctgatcttctctgagcatttcaatacgcgctaccctgattgtcgctgcctcaactttggttcctggtttttgttttccttcccggttgctctaattcttctacttttgtcttggatttggctccagtggctctacctaggattcgactttaagatgttcaagtgtggcaaaaccaaaacactcaaagaaaaagcttgtgcaaaggtgatcaaacaagaatatgaaaaacttgggccaatgaggtatcaagaaatcgtgaccttggtgatcttcattgtaatggccttgctctggttcagtcgggacccaggctttgtcactggttggtcggtcctgttttcagagtacccgggttatgttacagattcaacggttgctttagtagcaggaatccttttctttctaattccagctaagaaagtgacaaaaatgacatctgcaggagaaattattgcttttgattacactcctctgattacttggaaagaattccagtcattcatgccctgggacatagccattcttgttggtggaggctttgccctggcagatggctgtcaggtatcaggactatctaactggatagggagtaaattatctcctctaggttcattaccagtttggctaataattctgatatcttccttgattgtcacatctttgacagaggtagccagcaacccagctaccattaccattctgttcccaatattatctcctttggctgaagccattcaagtgaaccctcttcagattttgctgccttccactctctgtacctcatttgcatttcttctgccagttgcaaatccacccaatgctattgttttttcctatggccacctgaaagtcattgacatggttaaagctggacttggagtgaatattttgggtgttgctgtggtgatgctaggcatgttcacctggatcgagccgatgtttaacctccacgagtatccctcctgggcttctaactttgttaatcagaccatgcca

SEQ ID NO: 6 - protein, 594 aa, Mus musculus
MKLLNYALVYRRFLLVVFTILVFLPLPLIIRTKEAQCAYILFVIAIFWITEALPLSITALLPGLMFPMFGIMRSSQVASAYFKDFHLLLIGVICLATSIEKWNLHKRIALRMVMMVGVNPAWLTLGFMSSTAFLSMWLSNTSTAAMVMPIVEAVAQQIISAEAEAEATQMTYFNESAAHGLDIDETVIGQETNEKKEKTKPAPGSSHDKGKVSRKMETEKNAVTGAKYRSRKDHMMCKLMCLSVAYSSTIGGLTTITGTSTNLIFSEHFNTRYPDCRCLNFGSWFLFSFPVALILLLLSWIWLQWLYLGFDFKMFKCGKTKTLKEKACAKVIKQEYEKLGPMRYQEIVTLVIFIVMALLWFSRDPGFVTGWSVLFSEYPGYVTDSTVALVAGILFFLIPAKKVTKMTSAGEIIAFDYTPLITWKEFQSFMPWDIAILVGGGFALADGCQVSGLSNWIGSKLSPLGSLPVWLIILISSLIVTSLTEVASNPATITILFPILSPLAEAIQVNPLQILLPSTLCTSFAFLLPVANPPNAIVFSYGHLKVIDMVKAGLGVNILGVAVVMLGMFTWIEPMFNLHEYPSWASNFVNQTMP

SEQ ID NO: 7 - DNA, 1875 bp, Mus musculus
  source 1..1875: mol_type = other DNA
atgggcttgctgcagggccttctccaagcgaggaagctgctattggtcatctgtgtgccgctgctgctgctgcctctgcccaccatctaccccaccagcgaagctgcgtgtgcttacgtgttgcttgtgaccgctgtgtactgggtgtctgaggccgtgcctctcggagctgcagccctcgtgcctgccttcctctacccgttcttcggtgttctccgatccagtgaggtggcggctgagtacttcaagaacaccacgctgctgttggtgggcgtcatctgtgtggcagcagctgtggagaagtggaatttacacaagcgcatcgccctgcgcatggtcttgatggcgggagccaagcctggcatgctcctgctgtgtttcatgtgctgtaccaccatgctgtccatgtggctctccaacacctccaccaccgccatggtgatgcccatcgtggaggcggtgctgcaggagctcatcaacgctgaggaggaacagctggcagccggcactgaggaggctgagctcatgggtcttgatgtaaacaacagacagacgtccatggaactcatctttgtcaatgaagacacgtcggctgcagacgtcacttctctgatgcagagcaagaatctgaatggtgtgcccatggtcaccaaatccatcaacacagcaaaccaacagcaacagaagaagcagcagccgtcacaggaaaagccaggagtcccaacccccagctccaagacccaagaactgaacaagaagaagtacagatcccaccatgaccaaatgatctgcaagtgcctttctctgagcatttcctatgctgccaccattggtggcctgaccaccatcataggcacctccaccagcctcattttcttagagcatttcaacaaccagtacccggcggcagaggtggtgaacttcggaacctggttcctcttcagcttccccatctcgctcatcatgctggtggtcagctggttctggatccactggctcttcctgggctgcaacttcaaagagacctgctctctgagcaagaagaagaagaccaggagggaggagctatcagagaagaggatccgggaggaatatgagaagctaggagacattagctaccctgagatggtgaccgcctttttcttcattctgatgacggtgctgtggtttacccgggagcccggctttgtccctggctgggattctttctttgaaaagaaaggctaccgcactgatgccacagtctctgtattccttggcttcctgcttttcctcattccagcaaagaagccctgctttgggaaaaagagtgatggaacaggccaggaagcctccaaagggatagagcccatcatcacgtggaaggacttccagaaaactatgccctgggagattgtcattctggcgggaggaggctacgccctggcttctggcagcaagagctctggcctctccacatggatcgggcaccagatgctgtccctgagcagcctcccgccatgggctatcaccctgctggcgtgtgtcctggtgtccattgtcacggagtttgtcagcaatccagccaccatcaccatcttcctgcccatcctttgtaccctgtccgaaacgctgcacatcaatccactgtacaccctggtccccgttaccatgtcaatctccttcgctgtgatgctgcccgtgggcaacccccccaacgccatcgtcttcagctacgggcactgccagatcaaagacatggtgaaagccggtctgggagtcaatgtcatcggtctggtgatcgtgatggtggccatcaatacttggggagttagcctcttccacttggatgctttcccagcctgggctaaggtcagcaacatcactgatcagacc

SEQ ID NO: 8 - protein, 625 aa, Mus musculus
MGLLQGLLQARKLLLVICVPLLLLPLPTIYPTSEAACAYVLLVTAVYWVSEAVPLGAAALVPAFLYPFFGVLRSSEVAAEYFKNTTLLLVGVICVAAAVEKWNLHKRIALRMVLMAGAKPGMLLLCFMCCTTMLSMWLSNTSTTAMVMPIVEAVLQELINAEEEQLAAGTEEAELMGLDVNNRQTSMELIFVNEDTSAADVTSLMQSKNLNGVPMVTKSINTANQQQQKKQQPSQEKPGVPTPSSKTQELNKKKYRSHHDQMICKCLSLSISYAATIGGLTTIIGTSTSLIFLEHFNNQYPAAEVVNFGTWFLFSFPISLIMLVVSWFWIHWLFLGCNFKETCSLSKKKKTRREELSEKRIREEYEKLGDISYPEMVTAFFFILMTVLWFTREPGFVPGWDSFFEKKGYRTDATVSVFLGFLLFLIPAKKPCFGKKSDGTGQEASKGIEPIITWKDFQKTMPWEIVILAGGGYALASGSKSSGLSTWIGHQMLSLSSLPPWAITLLACVLVSIVTEFVSNPATITIFLPILCTLSETLHINPLYTLVPVTMSISFAVMLPVGNPPNAIVFSYGHCQIKDMVKAGLGVNVIGLVIVMVAINTWGVSLFHLDAFPAWAKVSNITDQT

SEQ ID NO: 9 - DNA, 1872 bp, Homo sapiens
  source 1..1872: mol_type = other DNA
atggagatccccgggagcttgtgcaagaaagtcaagctgagcaataacgcgcagaactggggaatgcagagagcaaccaatgtcacctaccaagcccatcatgtcagcaggaacaagagaggtcaggtggtggggaccagaggtggctttcgtggttgcacagtttggctaacaggcttgtctggagcgggaaagactactgtgagcatggccttggaggagtacctggtttgtcatggtattccatgctacactctggatggtgacaatattcgtcaaggtctcaataaaaatcttggctttagtcctgaagacagagaagagaatgttcgacgcatcgcagaagttgctaaactgtttgcagatgctggcttagtgtgcatcacaagtttcatatcaccttacactcaggatcgcaacaatgcaaggcaaattcatgaaggtgcaagtttaccgttttttgaagtatttgttgatgctcctctgcatgtttgtgaacagagggatgtcaaaggactctacaaaaaagcccgggcaggagaaattaaaggtttcactgggatcgattctgaatatgaaaagccagaggcccctgagttggtgctgaaaacagactcctgtgatgtaaatgactgtgtccagcaagttgtggaacttctacaggaacgggatattgtacctgtggatgcatcttatgaagtaaaagaactatatgtgccagaaaataaacttcatttggcaaaaacagatgcggaaacattaccagcactgaaaattaataaagtggatatgcagtgggtgcaggttttggcagaaggttgggcaaccccattgaatggctttatgagagagagggagtacttgcagtgccttcattttgattgtcttctggatggaggtgtcattaacttgtcagtacctatagttctgactgcgactcatgaagataaagagaggctggatggctgtacagcatttgctctgatgtatgagggccgccgtgtggccattcttcgcaatccagagttttttgagcacaggaaagaggagcgctgtgccagacagtggggaacgacatgcaagaaccacccctatattaagatggtgatggaacaaggagattggctgattggaggagatcttcaagtcttggatcgagtttattggaatgatggtcttgatcagtatcgtcttactcctactgagctaaagcagaaatttaaagatatgaatgctgatgctgtctttgcatttcaactacgcaacccagtgcacaatggacatgccctgttaatgcaggatacccataagcaacttctagagaggggctaccggcgccctgtcctcctcctccaccctctgggtggctggacaaaggatgacgatgttcctttgatgtggcgtatgaagcagcatgctgcagtgttggaggaaggagttctgaatcctgagacgacagtggtggccatcttcccatctcccatgatgtatgctggaccaactgaggtccagtggcattgcagagcacggatggttgcaggagccaacttttacattgttggacgagaccctgctggcatgcctcatccagaaacagggaaggatctttatgagccaagtcatggtgccaaagtgctgacgatggcccctggtttaatcactttggaaatagttccctttcgagttgcagcttacaacaagaaaaagaagcgtatggactactatgactctgaacaccatgaagactttgaatttatttcaggaacacgaatgcgcaaacttgctcgagaaggccagaaaccacctgaaggtttcatggctcccaaggcttggaccgtgctgacagaatactacaaatccttggagaaagct

SEQ ID NO: 10 - protein, 624 aa, Homo sapiens
MEIPGSLCKKVKLSNNAQNWGMQRATNVTYQAHHVSRNKRGQVVGTRGGFRGCTVWLTGLSGAGKTTVSMALEEYLVCHGIPCYTLDGDNIRQGLNKNLGFSPEDREENVRRIAEVAKLFADAGLVCITSFISPYTQDRNNARQIHEGASLPFFEVFVDAPLHVCEQRDVKGLYKKARAGEIKGFTGIDSEYEKPEAPELVLKTDSCDVNDCVQQVVELLQERDIVPVDASYEVKELYVPENKLHLAKTDAETLPALKINKVDMQWVQVLAEGWATPLNGFMREREYLQCLHFDCLLDGGVINLSVPIVLTATHEDKERLDGCTAFALMYEGRRVAILRNPEFFEHRKEERCARQWGTTCKNHPYIKMVMEQGDWLIGGDLQVLDRVYWNDGLDQYRLTPTELKQKFKDMNADAVFAFQLRNPVHNGHALLMQDTHKQLLERGYRRPVLLLHPLGGWTKDDDVPLMWRMKQHAAVLEEGVLNPETTVVAIFPSPMMYAGPTEVQWHCRARMVAGANFYIVGRDPAGMPHPETGKDLYEPSHGAKVLTMAPGLITLEIVPFRVAAYNKKKKRMDYYDSEHHEDFEFISGTRMRKLAREGQKPPEGFMAPKAWTVLTEYYKSLEKA

SEQ ID NO: 11 - DNA, 1863 bp, Mus musculus
  source 1..1863: mol_type = other DNA
atgtctgcaaatttcaaaatgaaccataaaagagaccagcaaaaatccaccaatgtggtctaccaggcccatcatgtgagcaggaacaagagaggacaagtggttggaaccaggggaggattccgaggatgtaccgtgtggctaacaggtctctctggtgctgggaaaacaaccataagctttgctttggaagagtaccttgtatctcacgccatcccatgttactctctggatggggacaatgtccgtcatggccttaataagaacctgggattctctgcgggggaccgagaagagaatatccgccggatcgcggaggtggccaggctctttgccgacgccggcctggtttgcatcaccagctttatctctccttttgcaaaggatcgtgagaatgcccgaaaaatccacgaatcagcaggactcccattctttgagatctttgtagatgcgcctttaaatatctgtgaaagccgagacgtaaaaggactctacaaacgagcccgagcaggagagattaaagggtttacaggcatcgattctgactatgagaaacctgaaactccagagtgtgtgctgaagaccaacctgtcttcagtaagcgactgtgtgcaacaggtggtggaacttttgcaggagcagaacattgtaccccacaccaccatcaaaggcatccacgaactctttgtgccagaaaacaaagtcgatcaaatccgagctgaggcagagactctcccatcactaccaattaccaagctggatctgcagtgggtgcagattctgagtgaaggctgggccactcccctcaaaggctttatgcgggagaaggagtacttgcaaactctacacttcgacactctactggacggcgtggttccccgtgatggagtcatcaacatgagtattcccattgtattgcccgtttctgcggatgacaaggcacggctcgaagggtgcagcaaatttgccttgatgtacgaaggtcggagggtcgctctattacaggaccctgaattctatgagcataggaaagaggagcgctgttctcgtgtgtggggaacagccactgcaaagcacccccatatcaaaatggtgatggaaagtggggactggcttgttggtggagacctacaggtgctagagagaataaggtgggacgatgggctggaccaataccgccttacgcctctggagctcaaacagaagtgtaaagacatgaatgctgatgccgtgtttgcattccagttgcgcaatcctgtccacaatggtcatgccctcctgatgcaggacacccgccgcaggctcctggagaggggttacaagcacccagtcctcctgctccaccctcttgggggctggaccaaggacgatgacgtacctctggaatggaggatgaaacagcatgcagctgtactggaggaaagggtcctggatcccaagtcaactattgttgccatctttccatctcctatgttatatgctggtcccacagaggtccagtggcattgcagatgccggatgattgcaggagccaatttctacattgtgggtagggatcccgcaggaatgccccatcctgagacaaagaaagacctatatgaacccacccacgggggcaaggtcttgagtatggcccctggccttacctctgtggaaataattccgttccgagtggctgcctacaataaaattaaaaaggccatggacttttatgatccagcaaggcacgaggagtttgacttcatctcaggaactcgcatgaggaagctcgcccgggaaggagaagatcccccagatggcttcatggccccgaaagcgtggaaagtgttgacagattactacaggtctctggagaagaccaac

SEQ ID NO: 12 - protein, 621 aa, Mus musculus
MSANFKMNHKRDQQKSTNVVYQAHHVSRNKRGQVVGTRGGFRGCTVWLTGLSGAGKTTISFALEEYLVSHAIPCYSLDGDNVRHGLNKNLGFSAGDREENIRRIAEVARLFADAGLVCITSFISPFAKDRENARKIHESAGLPFFEIFVDAPLNICESRDVKGLYKRARAGEIKGFTGIDSDYEKPETPECVLKTNLSSVSDCVQQVVELLQEQNIVPHTTIKGIHELFVPENKVDQIRAEAETLPSLPITKLDLQWVQILSEGWATPLKGFMREKEYLQTLHFDTLLDGVVPRDGVINMSIPIVLPVSADDKARLEGCSKFALMYEGRRVALLQDPEFYEHRKEERCSRVWGTATAKHPHIKMVMESGDWLVGGDLQVLERIRWDDGLDQYRLTPLELKQKCKDMNADAVFAFQLRNPVHNGHALLMQDTRRRLLERGYKHPVLLLHPLGGWTKDDDVPLEWRMKQHAAVLEERVLDPKSTIVAIFPSPMLYAGPTEVQWHCRCRMIAGANFYIVGRDPAGMPHPETKKDLYEPTHGGKVLSMAPGLTSVEIIPFRVAAYNKIKKAMDFYDPARHEEFDFISGTRMRKLAREGEDPPDGFMAPKAWKVLTDYYRSLEKTN

SEQ ID NO: 13 - DNA, 1293 bp, Mus musculus
  source 1..1293: mol_type = other DNA
atggacgccagatggtgggcagtagtggtactcgccacactcccttccttgggagcaggtggagagtcacccgaagcccctccgcagtcctggacacagctgtggctcttccgcttcttgttgaatgtagcgggctatgccagctttatggtacctggctacctcctggtgcagtacttaagacggaagaactacctggagacaggcaggggtctctgcttccccctggtgaaagcctgtgtgtttggcaatgagcccaaggctcctgatgaggttctcctggctccgcggacagagacagcggaatccaccccgtcttggcaggtcctgaagctggtcttctgtgcctcgggtctccaggtgtcctatctgacttggggcatactgcaggaaagagtgatgactggcagctacggggccacagccacatcaccaggagagcatttcacagactcccagtttctggtgctgatgaaccgtgtgctggcgctggttgtggcaggcctctactgtgtcctgcgcaagcagccccgtcatggtgcacccatgtaccggtactcctttgccagtctgtcaaatgtgcttagcagctggtgccagtatgaagcacttaagttcgtcagcttccctacccaggtgctggcgaaggcctccaaggtgatccctgtcatgatgatgggaaagctggtgtcccggcgcagctatgaacactgggaatacctgactgccggcctcatctccattggagtgagcatgtttcttctatccagtggaccagagcctagaagctctccagccaccacactctctggcttggtcctactggcaggctatattgctttcgacagcttcacctcaaattggcaggatgccctgtttgcctataagatgtcatcggtgcagatgatgtttggggtcaatttattctcctgtcttttcacagtaggctcactactggaacagggggccctactggagggggcacgcttcatggggcggcacagtgagtttgcgctccatgctctcctcctctccatctgctccgcctttgggcagctcttcatcttctacaccattggacaatttggagctgctgtcttcactatcatcatgactctacgccaggctattgccatcctcctctcctgcctcctctatggccatactgtcactgtggtggggggactgggagtagctgtggtcttcactgccctcctactcagagtctatgcccggggccggaagcagcggggaaagaaggctgtgcccactgagcccccagtacagaaggtg

SEQ ID NO: 14 - protein, 431 aa, Mus musculus
MDARWWAVVVLATLPSLGAGGESPEAPPQSWTQLWLFRFLLNVAGYASFMVPGYLLVQYLRRKNYLETGRGLCFPLVKACVFGNEPKAPDEVLLAPRTETAESTPSWQVLKLVFCASGLQVSYLTWGILQERVMTGSYGATATSPGEHFTDSQFLVLMNRVLALVVAGLYCVLRKQPRHGAPMYRYSFASLSNVLSSWCQYEALKFVSFPTQVLAKASKVIPVMMMGKLVSRRSYEHWEYLTAGLISIGVSMFLLSSGPEPRSSPATTLSGLVLLAGYIAFDSFTSNWQDALFAYKMSSVQMMFGVNLFSCLFTVGSLLEQGALLEGARFMGRHSEFALHALLLSICSAFGQLFIFYTIGQFGAAVFTIIMTLRQAIAILLSCLLYGHTVTVVGGLGVAVVFTALLLRVYARGRKQRGKKAVPTEPPVQKV

SEQ ID NO: 15 - DNA, 1107 bp, Mus musculus
  source 1..1107: mol_type = other DNA
atggacctcaagttcaacaactccaggaagtatgtctccatctcagtgccctccaaaacgcaggccatgtcgccgcatatcaagtcagttgaggacgtcgtggtgctgggcgtgaacctcagcaagtttagcaagctcacacagtttctcatctgtgtggctggagtttttgtattttacctaatttatggatacttacaggagctgatcttttcagtggaaggctttaagccttacggctggtaccttaccttagtgcagtttgccttttactccgtatttggcctaatcgagctacagctcactcaggacagaaggagaagaataccaggaaaaacctacatgctaatagcttttctaactgtgggtactatgggcttatcaaacacttccttgggctacctgaattacccaacccaagtcatcttcaagtgctgcaaactgattcctgttatgctaggaggagtttttattcaaggaaagcggtacaaccttgcagatgtgtctgccgcagtgtgcatgagcctgggcctgatctggtttaccctcgctgacagcacaattgcaccaaactttaatctgacaggtgtgatgcttatctccctggcgctgtgtgcagacgctgtcattgggaacgttcaggagaaggccatgaaactgcacaatgcttccaactcggagatggttttgtattcctactcaatcgggtttgtgtacattttgctgggactgtcatgcactagtggactgggccctgcagtggcattttgttcaaagaaccctgttgggacttacggctacgccttcctgttttccctcactggatactttgggatctcctttgttctggccctcattaaaatcttcggtgcacttctggctgtaacagtgacaacaggaaggaaagcaatgaccgttgtcctgtccttcctgttctttgctaagccgttcacttttcagtatatatggtctggcttgttagttgtccttggcatatttctcaatgtttacagcaaaaatatggataaaataagattgccatcggtgtacaacatgataaagaaagccatggacatgaaaaagtcaaggacgttggcacagactgtg

SEQ ID NO: 16 - protein, 369 aa, Mus musculus
MDLKFNNSRKYVSISVPSKTQAMSPHIKSVEDVVVLGVNLSKFSKLTQFLICVAGVFVFYLIYGYLQELIFSVEGFKPYGWYLTLVQFAFYSVFGLIELQLTQDRRRRIPGKTYMLIAFLTVGTMGLSNTSLGYLNYPTQVIFKCCKLIPVMLGGVFIQGKRYNLADVSAAVCMSLGLIWFTLADSTIAPNFNLTGVMLISLALCADAVIGNVQEKAMKLHNASNSEMVLYSYSIGFVYILLGLSCTSGLGPAVAFCSKNPVGTYGYAFLFSLTGYFGISFVLALIKIFGALLAVTVTTGRKAMTVVLSFLFFAKPFTFQYIWSGLLVVLGIFLNVYSKNMDKIRLPSVYNMIKKAMDMKKSRTLAQTV

SEQ ID NO: 17 - DNA, 1854 bp, Mus musculus
  source 1..1854: mol_type = other DNA
atgcgttgtttggcagctcgggtcaactataagactttgattatcatctgtgcgctattcactttggtcacagtacttttgtggaataagtgttccagcgacaaagcaatccagtttcctcggcacttgagtagtggattcagagtggatggattagaaaaaagatcagcagcatctgaaagtaaccactatgccaaccacatagccaaacagcagtcagaagaggcatttcctcaggaacaacagaaggcaccccctgttgttgggggcttcaatagcaacgggggaagcaaggtgttagggctcaaatatgaagagattgactgtctcataaacgatgagcacaccattaaagggagacgagaggggaatgaagttttccttccattcacttgggtagagaaatactttgatgtttatggaaaagtggtccagtatgacggctatgatcgatttgaattctctcatagctattccaaagtctatgcacagagatcaccttatcaccctgacggtgtgtttatgtcctttgaaggctacaatgtggaagtccgagacagagtcaaatgtataagtggagttgaaggtgtgccattatctacccagtgggggcctcaaggctatttctacccaatccagattgcacagtatgggctaagtcattacagcaagaatctaaccgagaaaccccctcacatagaagtatatgaaacagcagaagacagggacagaaacatcagacctaatgaatggactgtgcccaaggggtgcttcatggccagtgtggcagacaagtctagatccaccaatgttaaacagtttattgctccagaaaccagtgaaggtgtgtctttgcagctgggaaacacaaaagacttcattatttcatttgacctcaagcttttaacaaatgggagtgtgtctgtggttctggagaccacagaaaagaatcagctcttcactgtgcattatgtctcaaacacccagctgattgctttcagagacagggacatatactacggcattgggcccagaacttcatggagtacagttaccagagacctggtcactgacctcaggaaaggagtgggcctttctaacacaaaagctgtcaagccaaccaaaatcatgcccaaaaaggtggttaggttgattgcaaaagggaagggattcctggacaacattaccatctcaaccacagcccacatggctgcattctttgctgcaagtgactggctagtgaggaaccaggatgagaaaggtggctggccaattatggtgacccggaagttaggggaagggtttaaatctttagaaccaggatggtactctgccatggcacaagggcaagccatctctaccttagtcagggcctatcttctaacgaaagactatgtattcctcagttcagctttaagggcaacagccccatacaagtttccgtcagagcagcatggagttaaagccgtgttcatgaataaacatgactggtatgaagaatatccaaccacacctagctcttttgttttaaatggctttatgtattctttaattgggctgtatgacctaaaagaaacagcaggggagacacttgggaaagaagcaaggtccttgtacgagcgcggcatggaatctcttaaagccatgctgcccttgtatgatactggctccgggaccatctatgacctccgccacttcatgcttggcattgctcccaacctggcccgctgggactatcacaccacccacattaaccagctgcagctgctcagcaccatcgatgagtccccaatcttcaaagaatttgtcaagaggtggaaaagctaccttaaaggcagtagggcaaagcacaac

SEQ ID NO: 18 - protein, 618 aa, Mus musculus
MRCLAARVNYKTLIIICALFTLVTVLLWNKCSSDKAIQFPRHLSSGFRVDGLEKRSAASESNHYANHIAKQQSEEAFPQEQQKAPPVVGGFNSNGGSKVLGLKYEEIDCLINDEHTIKGRREGNEVFLPFTWVEKYFDVYGKVVQYDGYDRFEFSHSYSKVYAQRSPYHPDGVFMSFEGYNVEVRDRVKCISGVEGVPLSTQWGPQGYFYPIQIAQYGLSHYSKNLTEKPPHIEVYETAEDRDRNIRPNEWTVPKGCFMASVADKSRSTNVKQFIAPETSEGVSLQLGNTKDFIISFDLKLLTNGSVSVVLETTEKNQLFTVHYVSNTQLIAFRDRDIYYGIGPRTSWSTVTRDLVTDLRKGVGLSNTKAVKPTKIMPKKVVRLIAKGKGFLDNITISTTAHMAAFFAASDWLVRNQDEKGGWPIMVTRKLGEGFKSLEPGWYSAMAQGQAISTLVRAYLLTKDYVFLSSALRATAPYKFPSEQHGVKAVFMNKHDWYEEYPTTPSSFVLNGFMYSLIGLYDLKETAGETLGKEARSLYERGMESLKAMLPLYDTGSGTIYDLRHFMLGIAPNLARWDYHTTHINQLQLLSTIDESPIFKEFVKRWKSYLKGSRAKHN

SEQ ID NO: 19 - DNA, 1668 bp, Homo sapiens
  source 1..1668: mol_type = other DNA
atgcctgccctggcatgcctccggaggctgtgtcggcacgtgtccccgcaggctgtccttttcctgctgcaagtcgtctgccagttcagcgttttcatctcggcctactacctatatggctggaagcgaggcctggagccctcggcggatgcccccgagcctgactgcggggacccgccgcctgtggcccccagtcgcctgctgccactcaagcctgtgcaggcagccaccccttcccgcacagacccgttggtgctggtctttgtggagagcctctactcgcaactgggccaggaggtggtggccatcctggagtccagccgcttcaaataccgcacagagattgcgccgggcaagggtgacatgcccacgctcactgacaagggccgtggccgcttcgccctcatcatctatgagaacatcctcaagtatgtcaacctggacgcctggaaccgggagctgctggacaagtactgtgtggcctacggcgtgggcatcattggcttcttcaaggccaatgagaacagcctgctgagtgcgcagctcaagggcttccccctgttcctgcactcaaacctgggcctgaaggactgcagcatcaaccccaagtccccgctgctctacgtgacgcgacctagcgaggtggagaaaggtgtgctccccggcgaggactggacggttttccagtcaaatcactccacctatgagccagtgctgctggccaagacgcgctcgtctgagtccatcccacacctgggcgcagacgccggcctgcatgctgcactgcacgccactgtggtccaggacctgggcctgcacgacggcatccagcgcgtgctgtttggcaacaacctgaacttctggctgcacaagcttgtcttcgtggatgccgtggccttcctcacggggaagcgcctctccctgccattggaccgctacatcctggtggacattgatgacatcttcgtgggcaaggagggcacacgcatgaaggtggaggacgtgaaggccctgtttgacacacagaacgaactacgcgcacacatcccaaacttcaccttcaacctgggctactcagggaaattcttccagacaggtaccaatgctgaggacgctggggatgatctgctgctgtcgtatgtgaaggagttctggtggttcccccacatgtggagccacatgcagccccaccttttccacaaccagtccgtgttggccgagcagatggccttgaacaagaagttcgctgtcgagcatggcattcccacagacatggggtatgcagtggcgccccaccactcgggcgtgtaccccgtgcacgtgcagctgtacgaggcttggaagcaggtgtggagcatccgcgtgaccagcacggaggagtacccccacctgaagccagcccgctaccgccgtggcttcatccacaatggcatcatggttctcccacggcagacctgcggcctcttcacacacaccatcttctacaacgagtaccctggcggctccagtgagctggacaagatcatcaacgggggcgagctcttcctcaccgtgctcctcaatcctgtgagtgctccacagcccatggctgctggtgagaaggggctgctgcacagcctgtctgcagctgacacaggcttcctggagccaggaaagggtggagaggca

SEQ ID NO: 20 - protein, 556 aa, Homo sapiens [sequence omitted]

SEQ ID NO: 21 - DNA, 2649 bp, Homo sapiens
  source 1..2649: mol_type = other DNA
atgctccagttgtggaaggtggtacgcccagctcggcagctggaactgcaccgcctcatactgctgctgatcgctttcagcctgggctccatgggcttcctggcttattatgtgtccaccagccctaaggccaaggaacccttgcccctgcccttgggagactgcagcagcggtggggcagctggtcctggccctgcacggcctccagttccacctcggccccccaggcctccagagacagctcgaactgaacccgtggtccttgtgtttgtggagagtgcatactcacagctggggcaggaaattgtggccatcctggagtctagtcgttttcgttatagcactgagttggcacctggccgaggggacatgcccacattgactgataatacccatggccgctatgtcttggtcatttatgagaacctgctcaagtatgtcaacctggatgcctggagtcgggaactgctagaccggtactgcgtggagtatggtgtgggcatcattggctttttccgagcccacgagcacagcctactgagcgcccagctcaagggctttcccctttttttacactcaaacttggggctccgggactaccaagtgaatccttctgccccgctactgcatctcacacgccccagccgcctagaaccagggccactgcctggtgatgactggaccatcttccaatccaatcatagtacatatgaaccagtgcttcttgccagccttcggccagctgagcccgcagtgccaggaccagttcttcgtcgggcccggcttcccactgtggtacaggacctggggcttcatgatggcatccagcgggtgctctttggacatggcctttccttctggctccacaaacttatcttcgttgatgctgttgcatacctcactggcaagcgcctctgcctggaccttgaccgctacatcttggtagacatcgatgacatctttgtgggcaaggaagggacccgcatgaaggtggctgatgttgaggctctgttgaccacccagaacaaactcaggaccttagttcccaacttcaccttcaacttgggcttctcgggcaagttctatcatactgggacagaggaggaggatgcaggggacgacatgctgctgaagcaccgcaaagagttctggtggttcccccacatgtggagccacatgcagccacacctgttccacaatcgctccgtgctggctgaccagatgaggctcaacaaacagtttgctctggagcatgggattcccacggacctggggtatgctgtggccccccaccactcgggtgtgtaccccatccacacgcagctctatgaggcctggaaatccgtgtggggcatccaggtgaccagcactgaggagtatccccatctccgccctgcccgctaccgccgtggcttcattcacaatggcattatggtgctgccccggcagacatgtggcctcttcactcacacaatcttctataatgagtatcctggaggctctcgtgaactagaccggagcatccgaggtggagagctctttctgacagtgctgcttaatccgatcagcatctttatgacccatctgtccaattatggaaatgaccggctgggcctatacacctttgagagcttggtgcgcttcctccagtgttggacacggctgcgcctacagacccttcctcctgtcccacttgcacagaagtactttgaacttttccctcaggagcgaagccccctttggcagaatccctgtgatgacaagaggcacaaagatatctggtccaaggagaaaacctgtgatcgtctcccgaagttcctcattgtgggaccccagaaaacagggactacagctattcacttcttcctgagcctgcacccagctgtaactagcagcttccctagccccagcacatttgaggagattcagttcttcaacagccctaattaccacaagggtattgactggtacatggatttcttccctgttccttccaatgccagcactgatttcctatttgaaaaaagtgccacctactttgactctgaagttgtaccacggcggggggctgccctcctgccacgagccaagatcatcacagtgctcaccaaccctgctgacagggcctactcctggtaccagcatcagcgagcccatggagacccagttgctctgaactataccttctatcaggtgatttcagcctcctcccagacccctctggcactacgctccctgcagaaccgctgtcttgtccctggctactattctacccatctacaacgctggctgacttactacccctctggacagttgctgattgtggatgggcaagagctgcgtaccaacccagcagcctcaatggagagcatccagaagttcctgggtatcacaccctttctgaactacacacggaccctcaggtttgatgatgataagggattttggtgccagggacttgaaggtggtaagactcgctgtctaggccggagcaaaggccggaggtatccagatatggacactgagtcccgtcttttccttacggattttttccggaaccataatttggagttgtcgaagctgctgagccggcttggacagccagtgccctcgtggcttcgggaagaactgcagcattccagtctgggc

SEQ ID NO: 22 - protein, 883 aa, Homo sapiens
MLQLWKVVRPARQLELHRLILLLIAFSLGSMGFLAYYVSTSPKAKEPLPLPLGDCSSGGAAGPGPARPPVPPRPPRPPETARTEPVVLVFVESAYSQLGQEIVAILESSRFRYSTELAPGRGDMPTLTDNTHGRYVLVIYENLLKYVNLDAWSRELLDRYCVEYGVGIIGFFRAHEHSLLSAQLKGFPLFLHSNLGLRDYQVNPSAPLLHLTRPSRLEPGPLPGDDWTIFQSNHSTYEPVLLASLRPAEPAVPGPVLRRARLPTVVQDLGLHDGIQRVLFGHGLSFWLHKLIFVDAVAYLTGKRLCLDLDRYILVDIDDIFVGKEGTRMKVADVEALLTTQNKLRTLVPNFTFNLGFSGKFYHTGTEEEDAGDDMLLKHRKEFWWFPHMWSHMQPHLFHNRSVLADQMRLNKQFALEHGIPTDLGYAVAPHHSGVYPIHTQLYEAWKSVWGIQVTSTEEYPHLRPARYRRGFIHNGIMVLPRQTCGLFTHTIFYNEYPGGSRELDRSIRGGELFLTVLLNPISIFMTHLSNYGNDRLGLYTFESLVRFLQCWTRLRLQTLPPVPLAQKYFELFPQERSPLWQNPCDDKRHKDIWSKEKTCDRLPKFLIVGPQKTGTTAIHFFLSLHPAVTSSFPSPSTFEEIQFFNSPNYHKGIDWYMDFFPVPSNASTDFLFEKSATYFDSEVVPRRGAALLPRAKIITVLTNPADRAYSWYQHQRAHGDPVALNYTFYQVISASSQTPLALRSLQNRCLVPGYYSTHLQRWLTYYPSGQLLIVDGQELRTNPAASMESIQKFLGITPFLNYTRTLRFDDDKGFWCQGLEGGKTRCLGRSKGRRYPDMDTESRLFLTDFFRNHNLELSKLLSRLGQPVPSWLREELQHSSLG

SEQ ID NO: 23 - DNA, 687 bp, Homo sapiens
  source 1..687: mol_type = other DNA
atggggctcctcaggattatgatgccgcccaagttgcagctgctggcggtggtggccttcgcggtggcgatgctcttcttggaaaaccagatccagaaactggaggagtcccgctcgaagctagaaagggctattgcaagacacgaagtccgagaaattgagcagcgacatacaatggatggccctcggcaagatgccactttagatgaggaagaggacatggtgatcatttataacagagttcccaaaacggcaagcacttcatttaccaatatcgcctatgacctgtgtgcaaagaataaataccatgtccttcatatcaacactaccaaaaataatccagtgatgtcattgcaagatcaggtgcgctttgtaaagaatataacttcctggaaagagatgaaaccaggattttatcatggacacgtttcttacttggattttgcaaaatttggtgtgaagaagaaaccaatttacattaatgtcataagggatcctattgagaggctagtttcttattattactttctgagatttggagatgattatagaccagggttacggagacgaaaacaaggagacaaaaagacctttgatgaatgtgtagcagaaggtggctcagactgtgctccagagaagctctggcttcaaatcccgttcttctgtggccatagctccgaatgctgg

SEQ ID NO: 24 - protein, 229 aa, Homo sapiens
MGLLRIMMPPKLQLLAVVAFAVAMLFLENQIQKLEESRSKLERAIARHEVREIEQRHTMDGPRQDATLDEEEDMVIIYNRVPKTASTSFTNIAYDLCAKNKYHVLHINTTKNNPVMSLQDQVRFVKNITSWKEMKPGFYHGHVSYLDFAKFGVKKKPIYINVIRDPIERLVSYYYFLRFGDDYRPGLRRRKQGDKKTFDECVAEGGSDCAPEKLWLQIPFFCGHSSECW

SEQ ID NO: 25 - DNA, 1203 bp, Mus musculus
  source 1..1203: mol_type = other DNA
atggttgagcgcgccagcaagttcgtgctggtggtggcgggctcggcgtgcttcatgctcatcctttaccagtacgcgggcccggggctgagtctgggcgcgcccggtggccgcgtgccccccgacgacctggatctcttccccacgccggacccacattacgagaaaaagtactacttcccggtgcgcgagctggagcgctcgctgcgcttcgacatgaagggcgacgacgtgatcgtcttcctgcacatccagaagaccggcggcaccaccttcggccgccacctagtgcagaacgtgcgcctcgaggtgccctgcgactgtcgcccgggccagaagaagtgcacctgctatcggcccaatcgccgcgagacctggctcttctctcgcttctccacgggctggagctgcgggctgcacgctgactggaccgaactcaccaactgtgtgcccggtgtgctagaccgccgcgacccagcaggtctgcgttcgcccagaaagttctactacatcaccctgctgcgagaccccgtatcccgctacctgagtgaatggcgacatgtacagcgtggggccacgtggaagacctccttgcacatgtgtgacgggcgcacaccgaccccagaggagctgccgccctgctacgagggcacagactggtcgggctgcacgttgcaggagttcatggattgcccctataacctggctaacaaccggcaggtgcgcatgctggccgacctcagcctggtgggctgctacaacctatctttcatccccgagagcaagcgggcccagttgctgctggagagcgccaagaagaacctgcgaggcatggccttcttcggcctcactgagttccagcgcaagacgcagtacctatttgagcggacgttcaacctcaagttcatccggccattcatgcaatacaacagcacgcgggcgggcggtgtggaggtggatgaggacactatccgccacatcgaggagctcaacgacctggacatgcagctgtatgactatgccaaggacctctttcagcagcgttaccagtacaagagacagctggagcgcagggaacagcgcctgcgcaatcgcgaagagcgcctcctgcaccgctccaaggaagcgctgccacgggaggacccagaagagccgggccgtgtgcccaccgaggactacatgagccatatcattgagaagtgg

SEQ ID NO: 26 - protein, 401 aa, Mus musculus
MVERASKFVLVVAGSACFMLILYQYAGPGLSLGAPGGRVPPDDLDLFPTPDPHYEKKYYFPVRELERSLRFDMKGDDVIVFLHIQKTGGTTFGRHLVQNVRLEVPCDCRPGQKKCTCYRPNRRETWLFSRFSTGWSCGLHADWTELTNCVPGVLDRRDPAGLRSPRKFYYITLLRDPVSRYLSEWRHVQRGATWKTSLHMCDGRTPTPEELPPCYEGTDWSGCTLQEFMDCPYNLANNRQVRMLADLSLVGCYNLSFIPESKRAQLLLESAKKNLRGMAFFGLTEFQRKTQYLFERTFNLKFIRPFMQYNSTRAGGVEVDEDTIRHIEELNDLDMQLYDYAKDLFQQRYQYKRQLERREQRLRNREERLLHRSKEALPREDPEEPGRVPTEDYMSHIIEKW

SEQ ID NO: 27 - DNA, 1398 bp, Mus musculus
  source 1..1398: mol_type = other DNA
atggatgagaaatctaacaagctgctgctggctttggtgatgctcttcctatttgcggtgatcgtcctccaatacgtgtgccccggcacagaatgccagctcctccgcctgcaggcgttcagctccccggtgccggatccgtaccgctcggaggatgagagttcggccaggtttgtaccccgctacaatttcagccgcggcgatctcctgcgcaaggtagacttcgacatcaagggcgatgacctgatcgtgttcctgcacatccagaagactgggggcaccacttttggccgtcacctggtgcgcaacatccagctggagcagccatgtgagtgccgcgtggggcagaagaaatgcacttgccaccggccgggtaagagggagacctggctcttctccaggttctccaccggctggagctgcgggctgcatgccgactggaccgagctcaccagctgcgtgccggcggtggtggatggcaagcgcgacgccaggctgagaccttccaggaacttccattacattaccatcctgagagacccagtgtcacggtacttgagtgaatggaggcatgtccagagaggagcaacttggaaagcatccctgcacgtctgtgatggaaggcccccaacctctgaagagctgcccagctgctacaccggtgatgactggtctggatgccctctcaaagagttcatggactgtccctataatctggccaacaaccgccaagttcgcatgctatctgacctgactctagtgggatgctacaacctctctgtcatgcctgaaaagcaaagaaacaaagtccttctggaaagtgccaaatccaatctgaagcacatggcgttctttggcctcactgagtttcagcgcaagacccagtacctgtttgagaagaccttcaacatgaactttatctcgccgtttacccagtataataccactagggcctctagtgttgagatcaatgaggaaatccaaaagcgtattgagggactgaattttctggatatggagttgtacagctatgctaaagacctctttctgcaaaggtatcagttcatgaggcagaaagaacatcaggatgccaggcggaagcgtcaggagcaacgcaaatttctgaagggaaggttccttcagacccatttccagagtcagagtcagggtcagagccagagccagagtccaggtcagaatctgagtcagaatccaaatcctaacccaaatcagaacctgactcagaacctgagtcacaatctgactccgagttcaaatcccaattcgacccagagggagaaccggggaagtcagaagcagggctcaggccagggacaaggtgatagcggcaccagcaatggcaccaatgactacatagggagcgtagagacatggcgc

SEQ ID NO: 28 - protein, 466 aa, Mus musculus
MDEKSNKLLLALVMLFLFAVIVLQYVCPGTECQLLRLQAFSSPVPDPYRSEDESSARFVPRYNFSRGDLLRKVDFDIKGDDLIVFLHIQKTGGTTFGRHLVRNIQLEQPCECRVGQKKCTCHRPGKRETWLFSRFSTGWSCGLHADWTELTSCVPAVVDGKRDARLRPSRNFHYITILRDPVSRYLSEWRHVQRGATWKASLHVCDGRPPTSEELPSCYTGDDWSGCPLKEFMDCPYNLANNRQVRMLSDLTLVGCYNLSVMPEKQRNKVLLESAKSNLKHMAFFGLTEFQRKTQYLFEKTFNMNFISPFTQYNTTRASSVEINEEIQKRIEGLNFLDMELYSYAKDLFLQRYQFMRQKEHQDARRKRQEQRKFLKGRFLQTHFQSQSQGQSQSQSPGQNLSQNPNPNPNQNLTQNLSHNLTPSSNPNSTQRENRGSQKQGSGQGQGDSGTSNGTNDYIGSVETWR

SEQ ID NO: 29 - DNA, 1410 bp, Mus musculus
  source 1..1410: mol_type = other DNA
atggatgaaaggttcaacaagtggctgctgacgccggtgctcactttcctcttcgtggtcatcatgtaccagtacgtgtccccttcctgcaccagctcatgcaccaacttcggggagcagctccgctccggggaggcccgcccgcccgctgtcccgagtcctgcccgacgggcacaagcaccccttgatgagtgggagcggcggccccagctgccaccgccaccccggggaccgcccgaggggtctcgtggggtcgcggctcccgaggatgaggatgaggatccgggggacccggaggaggaggaggaggaagaagaggaggagccggaccccgaggcgcccgaaaacggctccctgccccggttcgtgcctcgattcaacttcaccctcaaggacctgacccgcttcgtggacttcaacatcaaagggcgggatgtgatcgtgttcctgcacatccagaagaccgggggcaccacgttcggccggcacctggtgaagaacattcgtctggagcagccatgcagctgcaaagctggccagaaaaagtgcacctgccaccggccgggcaagaaggagacctggctcttctcccgcttctccaccggctggagctgcgggctgcatgccgactggaccgagctcaccaactgcgtgccggccatcatggagaagaaggattgtccccgcaaccacagccacaccaggaatttctactacatcacgatgttgcgggacccagtgtcacgttacctgagcgaatggaagcatgtccagagaggggccacatggaaaacctctctccatatgtgcgacggaaggagccccacaccagatgagctgcctacctgctatcctggggatgactggtctggggtcagcttgcgggagttcatggattgtagctacaacctggccaacaaccgccaagtgcgcatgctggccgacctcagcctggtgggctgctacaacttgactttcatgaatgagagcgagcggaacaccatcctgctgcagagcgctaagaacaatctgaagaatatggccttctttgggctcactgaattccagaggaagacacagtttctctttgagaggacattcaacctgaagttcatctctcctttcacacagttcaacatcacacgggcatccaacgtggacatcaatgatggagcccgtcagcacatcgaggagttgaatttcctggacatgcagctttatgagtatgcgaaggatctcttccagcaacgctaccatcacaccaaacagctagagcaccagagggaccgccaaaagaggcgcgaggagcggaggctgcagcgagagcacagagcacaccggtggcccaaagaggatagggccatggaggggactgtcactgaggactacaacagccaagtggtcagatgg

SEQ ID NO: 30 - protein, 470 aa, Mus musculus
MDERFNKWLLTPVLTFLFVVIMYQYVSPSCTSSCTNFGEQLRSGEARPPAVPSPARRAQAPLDEWERRPQLPPPPRGPPEGSRGVAAPEDEDEDPGDPEEEEEEEEEEPDPEAPENGSLPRFVPRFNFTLKDLTRFVDFNIKGRDVIVFLHIQKTGGTTFGRHLVKNIRLEQPCSCKAGQKKCTCHRPGKKETWLFSRFSTGWSCGLHADWTELTNCVPAIMEKKDCPRNHSHTRNFYYITMLRDPVSRYLSEWKHVQRGATWKTSLHMCDGRSPTPDELPTCYPGDDWSGVSLREFMDCSYNLANNRQVRMLADLSLVGCYNLTFMNESERNTILLQSAKNNLKNMAFFGLTEFQRKTQFLFERTFNLKFISPFTQFNITRASNVDINDGARQHIEELNFLDMQLYEYAKDLFQQRYHHTKQLEHQRDRQKRREERRLQREHRAHRWPKEDRAMEGTVTEDYNSQVVRW

SEQ ID NO: 31 - DNA, 933 bp, Mus musculus
  source 1..933: mol_type = other DNA
atgaccttgctgctcctgggtgcggtgctgctggtggcccagccccagcttgtgcattcccacccggctgctcctggcccggggctcaaacagcaggagcttctgaggaaggtgattattctcccagaggacaccggagaaggcacagcatccaatggttccacacagcagctgccacagaccatcatcattggggtgcgcaagggtggtacccgagccctgctagagatgctcagcctgcatcctgatgttgctgcagctgaaaacgaggtccatttctttgactgggaggagcattacagccaaggcctgggctggtacctcacccagatgcccttctcctcccctcaccagctcaccgtggagaagacacccgcctatttcacttcgcccaaagtgcctgagagaatccacagcatgaaccccaccatccgcctgctgcttatcctgagggacccatcagagcgcgtgctgtccgactacacccaggtgttgtacaaccaccttcagaagcacaagccctatccacccattgaggacctcctaatgcgggacggtcggctgaacctggactacaaggctctcaaccgcagcctgtaccatgcacacatgctgaactggctgcgttttttcccgttgggccacatccacattgtggatggcgaccgcctcatcagagaccctttccctgagatccagaaggtcgaaagattcctgaagctttctccacagatcaacgcctcgaacttctactttaacaaaaccaagggcttctactgcctgcgggacagtggcaaggaccgctgcttacacgagtccaaaggccgggcgcacccccaggtggatcccaaactacttgataaactgcacgaatactttcatgagccaaataagaaatttttcaagctcgtgggcagaacattcgactggcac

SEQ ID NO: 32 - protein, 311 aa, Mus musculus [sequence omitted]

SEQ ID NO: 33 - DNA, 1038 bp, Mus musculus
  source 1..1038: mol_type = other DNA
atgctattcaaacagcaggtgtggctgagacagaagctcctggtactgggaagccttgctgttgggagcctcctgtatctagttgccagagttgggagcttggataggctccagcccatttgccctgttgaaagtcgatttggtggtgcccacaaccaggctgagttgccactgcgggccctgcagtttaagagaggcttgctgcatgagttccggaagggcaattcttccaaggagcaggttcacctccatgacctggtccaacagctccccaaagccattatcattggggtgagaaaagggggcacaagggccctgctagagatgctcaacctccatcctgcagtggtcaaagcttcccaagagatccacttctttgacaatgatgagaattatgccaagggcattgagtggtacaggaaaaagatgcctttttcctaccctcagcaaatcacgattgaaaagagcccggcatatttcatcacagaagaggttccggaaaggatttacaagatgaactcatccatcaagctgttgatcattgtcagggagccgaccacaagagcaatttctgattatactcaggtgctagaggggaaggagcggaagaacaaaacctactataagtttgaaaaactggctatagaccctaatacctgtgaagtgaacacgaaatacaaggcggttaggaccagcatatacacaaaacatctggagcgctggttgaaatactttcccattgaacagtttcatatcgtagatggtgaccgtcttatcacagaacctctgccggaactacagctggtggaaaagttcttaaaccttcctccgaggataagtcagtacaatttatatttcaatgctaccagagggttttactgtctgagatttaacattatctttaataagtgcctggcgggcagcaaggggcgcatccatccagaggtagacccctccgtcattaccaaattgcgcaaattctttcatccttttaatcaaaaattttaccagatcactgggaggacattgaactggccc

SEQ ID NO: 34 - protein, 346 aa, Mus musculus
MLFKQQVWLRQKLLVLGSLAVGSLLYLVARVGSLDRLQPICPVESRFGGAHNQAELPLRALQFKRGLLHEFRKGNSSKEQVHLHDLVQQLPKAIIIGVRKGGTRALLEMLNLHPAVVKASQEIHFFDNDENYAKGIEWYRKKMPFSYPQQITIEKSPAYFITEEVPERIYKMNSSIKLLIIVREPTTRAISDYTQVLEGKERKNKTYYKFEKLAIDPNTCEVNTKYKAVRTSIYTKHLERWLKYFPIEQFHIVDGDRLITEPLPELQLVEKFLNLPPRISQYNLYFNATRGFYCLRFNIIFNKCLAGSKGRIHPEVDPSVITKLRKFFHPFNQKFYQITGRTLNWP

SEQ ID NO: 35 - DNA, 47 bp, synthetic construct
  misc_feature 1..47: note = Synthesized Sequence
  source 1..47: mol_type = other DNA
aaaatctagagccgccaccatggatgaaaggttcaacaagtggctgc

SEQ ID NO: 36 - DNA, 46 bp, synthetic construct
  misc_feature 1..46: note = Synthesized Sequence
  source 1..46: mol_type = other DNA
tttaaatcgcgattatcaccatctgaccacttggctgttgtagtcc

SEQ ID NO: 37 - DNA, 687 bp, Homo sapiens
  source 1..687: mol_type = other DNA
atgaggcgcgcggcgctctggctctggctgtgcgcgctggcgctgagcctgcagccggccctgccgcaaattgtggctactaatttgccccctgaagatcaagatggctctggggatgactctgacaacttctccggctcaggtgcaggtgctttgcaagatatcaccttgtcacagcagaccccctccacttggaaggacacgcagctcctgacggctattcccacgtctccagaacccaccggcctggaggctacagctgcctccacctccaccctgccggctggagaggggcccaaggagggagaggctgtagtcctgccagaagtggagcctggcctcaccgcccgggagcaggaggccaccccccgacccagggagaccacacagctcccgaccactcatcaggcctcaacgaccacagccaccacggcccaggagcccgccacctcccacccccacagggacatgcagcctggccaccatgagacctcaacccctgcaggacccagccaagctgaccttcacactccccacacagaggatggaggtccttctgccaccgagagggctgctgaggatggagcctccagtcagctcccagcagcagagggctctggggagcaggacttcacctttgaaacctcgggggagaatacggctgtagtggccgtggagcctgaccgc

SEQ ID NO: 38 - protein, 229 aa, Homo sapiens
MRRAALWLWLCALALSLQPALPQIVATNLPPEDQDGSGDDSDNFSGSGAGALQDITLSQQTPSTWKDTQLLTAIPTSPEPTGLEATAASTSTLPAGEGPKEGEAVVLPEVEPGLTAREQEATPRPRETTQLPTTHQASTTTATTAQEPATSHPHRDMQPGHHETSTPAGPSQADLHTPHTEDGGPSATERAAEDGASSQLPAAEGSGEQDFTFETSGENTAVVAVEPDR

SEQ ID NO: 39 - DNA, 2652 bp, Homo sapiens
  source 1..2652: mol_type = other DNA
atgctccagttgtggaaggtggtacgcccagctcggcagctggaactgcaccgcctcatactgctgctgatcgctttcagcctgggctccatgggcttcctggcttattatgtgtccaccagccctaaggccaaggaacccttgcccctgcccttgggagactgcagcagcggtggggcagctggtcctggccctgcacggcctccagttccacctcggccccccaggcctccagagacagctcgaactgaacccgtggtccttgtgtttgtggagagtgcatactcacagctggggcaggaaattgtggccatcctggagtctagtcgttttcgttatagcactgagttggcacctggccgaggggacatgcccacattgactgataatacccatggccgctatgtcttggtcatttatgagaacctgctcaagtatgtcaacctggatgcctggagtcgggaactgctagaccggtactgcgtggagtatggtgtgggcatcattggctttttccgagcccacgagcacagcctactgagcgcccagctcaagggctttcccctttttttacactcaaacttggggctccgggactaccaagtgaatccttctgccccgctactgcatctcacacgccccagccgcctagaaccagggccactgcctggtgatgactggaccatcttccaatccaatcatagtacatatgaaccagtgcttcttgccagccttcggccagctgagcccgcagtgccaggaccagttcttcgtcgggcccggcttcccactgtggtacaggacctggggcttcatgatggcatccagcgggtgctctttggacatggcctttccttctggctccacaaacttatcttcgttgatgctgttgcatacctcactggcaagcgcctctgcctggaccttgaccgctacatcttggtagacatcgatgacatctttgtgggcaaggaagggacccgcatgaaggtggctgatgttgaggctctgttgaccacccagaacaaactcaggaccttagttcccaacttcaccttcaacttgggcttctcgggcaagttctatcatactgggacagaggaggaggatgcaggggacgacatgctgctgaagcaccgcaaagagttctggtggttcccccacatgtggagccacatgcagccacacctgttccacaatcgctccgtgctggctgaccagatgaggctcaacaaacagtttgctctggagcatgggattcccacggacctggggtatgctgtggccccccaccactcgggtgtgtaccccatccacacgcagctctatgaggcctggaaatccgtgtggggcatccaggtgaccagcactgaggagtatccccatctccgccctgcccgctaccgccgtggcttcattcacaatggcattatggtgctgccccggcagacatgtggcctcttcactcacacaatcttctataatgagtatcctggaggctctcgtgaactagaccggagcatccgaggtggagagctctttctgacagtgctgcttaatccgatcagcatctttatgacccatctgtccaattatggaaatgaccggctgggcctatacacctttgagagcttggtgcgcttcctccagtgttggacacggctgcgcctacagacccttcctcctgtcccacttgcacagaagtactttgaacttttccctcaggagcgaagccccctttggcagaatccctgtgatgacaagaggcacaaagatatctggtccaaggagaaaacctgtgatcgtctcccgaagttcctcattgtgggaccccagaaaacagggactacagctattcacttcttcctgagcctgcacccagctgtaactagcagcttccctagccccagcacatttgaggagattcagttcttcaacagccctaattaccacaagggtattgactggtacatggatttcttccctgttccttccaatgccagcactgatttcctatttgaaaaaagtgccacctactttgactctgaagttgtaccacggcggggggctgccctcctgccacgagccaagatcatcacagtgctcaccaaccctgctgacagggcctactcctggtaccagcatcagcgagcccatggagacccagttgctctgaactataccttctatcaggtgatttcagcctcctcccagacccctctggcactacgctccctgcagaaccgctgtcttgtccctggctactattctacccatctacaacgctggctgacttactacccctctggacagttgctgattgtggatgggcaagagctgcgtaccaacccagcagcctcaatggagagcatccagaagttcctgggtatcacaccctttctgaactacacacggaccctcaggtttgatgatgataagggattttggtgccagggacttgaaggtggtaagactcgctgtctaggccggagcaaaggccggaggtatccagatatggacactgagtcccgtcttttccttacggattttttccggaaccataatttggagttgtcgaagctgctgagccggcttggacagccagtgccctcgtggcttcgggaagaactgcagcattccagtctgggctga

SEQ ID NO: 40 - protein, 883 aa, Homo sapiens; identical to SEQ ID NO: 22

SEQ ID NO: 41 - DNA, 936 bp, Mus musculus
  source 1..936: mol_type = other DNA
atgaccttgctgctcctgggtgcggtgctgctggtggcccagccccagcttgtgcattcccacccggctgctcctggcccggggctcaaacagcaggagcttctgaggaaggtgattattctcccagaggacaccggagaaggcacagcatccaatggttccacacagcagctgccacagaccatcatcattggggtgcgcaagggtggtacccgagccctgctagagatgctcagcctgcatcctgatgttgctgcagctgaaaacgaggtccatttctttgactgggaggagcattacagccaaggcctgggctggtacctcacccagatgcccttctcctcccctcaccagctcaccgtggagaagacacccgcctatttcacttcgcccaaagtgcctgagagaatccacagcatgaaccccaccatccgcctgctgcttatcctgagggacccatcagagcgcgtgctgtccgactacacccaggtgttgtacaaccaccttcagaagcacaagccctatccacccattgaggacctcctaatgcgggacggtcggctgaacctggactacaaggctctcaaccgcagcctgtaccatgcacacatgctgaactggctgcgttttttcccgttgggccacatccacattgtggatggcgaccgcctcatcagagaccctttccctgagatccagaaggtcgaaagattcctgaagctttctccacagatcaacgcctcgaacttctactttaacaaaaccaagggcttctactgcctgcgggacagtggcaaggaccgctgcttacacgagtccaaaggccgggcgcacccccaggtggatcccaaactacttgataaactgcacgaatactttcatgagccaaataagaaatttttcaagctcgtgggcagaacattcgactggcactga

SEQ ID NO: 42 - protein, 311 aa, Homo sapiens [sequence omitted]

SEQ ID NO: 43 - DNA, 1413 bp, Homo sapiens
  source 1..1413: mol_type = other DNA
atggatgaaaggttcaacaagtggctgctgacgccggtgctcactctcctcttcgtggtcatcatgtaccagtacgtgtccccctcctgcaccagctcctgcaccaacttcggggagcagccccgcgcgggggaggccggcccgcccgccgtcccgggtcccgcccgccgggctcaggcgccgccggaggagtgggagcggcggccccagttgcccccgccgccccgggggccccccgagggacctcggggggccgcggcgccggaggaggaggacgaggagcccggagacccccgggagggggaggaagaggaggaggaagacgagccggaccccgaggccccggaaaacggctccctgccccgattcgtgccgcgcttcaacttcagcctgaaggacctgacccgcttcgtggatttcaacatcaaagggcgcgacgtgatcgtgttcctccacatccagaagacggggggcaccactttcggccggcacctggtgaagaacatccggctggagcagccttgtagctgcaaagcgggtcagaagaagtgcacctgccaccggcctggcaagaaggagacgtggctcttctcccgcttctccaccggctggagctgcgggctgcacgccgactggacggagctcaccaactgcgtgccggccatcatggagaagaaggactgtccccgcaaccacagccacaccaggaatttctattacatcacaatgttacgggatccagtgtcacgttacctgagcgagtggaaacatgtccagagaggggccacttggaaaacctctcttcatatgtgtgatggaagaagccccaccccagatgagctgcctacctgctaccctggggatgactggtctggggtcagcttgcgggagtttatggattgcacctacaacctggctaacaatcgccaggtgcgcatgctggctgacctcagcctggtgggctgctataacttgactttcatgaacgagagtgaaagaaacaccatcctgttgcagagtgcaaagaacaacctgaagaacatggccttctttgggctcactgagttccagaggaagacacagtttctctttgagagaacattcaacctcaagttcatctcccccttcacacagttcaacatcacgcgggcttctaacgtggagatcaacgagggtgcccgccaacgcattgaggatctaaacttcctggacatgcagctttacgagtatgcaaaagatctcttccagcagcgctaccaccacaccaagcagctagagcaccagagggaccgccagaagcggcgggaggagcggaggctgcagcgagagcacagggaccaccagtggcccaaagaagatggggctgcagaagggactgtcaccgaggactacaacagccaggtggtgagatgg

SEQ ID NO: 44 - protein, 471 aa, Homo sapiens
MDERFNKWLLTPVLTLLFVVIMYQYVSPSCTSSCTNFGEQPRAGEAGPPAVPGPARRAQAPPEEWERRPQLPPPPRGPPEGPRGAAAPEEEDEEPGDPREGEEEEEEDEPDPEAPENGSLPRFVPRFNFSLKDLTRFVDFNIKGRDVIVFLHIQKTGGTTFGRHLVKNIRLEQPCSCKAGQKKCTCHRPGKKETWLFSRFSTGWSCGLHADWTELTNCVPAIMEKKDCPRNHSHTRNFYYITMLRDPVSRYLSEWKHVQRGATWKTSLHMCDGRSPTPDELPTCYPGDDWSGVSLREFMDCTYNLANNRQVRMLADLSLVGCYNLTFMNESERNTILLQSAKNNLKNMAFFGLTEFQRKTQFLFERTFNLKFISPFTQFNITRASNVEINEGARQRIEDLNFLDMQLYEYAKDLFQQRYHHTKQLEHQRDRQKRREERRLQREHRDHQWPKEDGAAEGTVTEDYNSQVVRW

SEQ ID NO: 45 - DNA, 921 bp, Homo sapiens
  source 1..921: mol_type = other DNA
atggccgcgctgctcctgggcgcggtgctgctggtggcccagccccagctagtgccttcccgccccgccgagctaggccagcaggagcttctgcggaaagcggggaccctccaggatgacgtccgcgatggcgtggccccaaacggctctgcccagcagttgccgcagaccatcatcatcggcgtgcgcaagggcggcacgcgcgcactgctggagatgctcagcctgcaccccgacgtggcggccgcggagaacgaggtccacttcttcgactgggaggagcattacagccacggcttgggctggtacctcagccagatgcccttctcctggccacaccagctcacagtggagaagacccccgcgtatttcacgtcgcccaaagtgcctgagcgagtctacagcatgaacccgtccatccggctgctgctcatcctgcgagacccgtcggagcgcgtgctatctgactacacccaagtgttctacaaccacatgcagaagcacaagccctacccgtccatcgaggagttcctggtgcgcgatggcaggctcaatgtggactacaaggccctcaaccgcagcctctaccacgtgcacatgcagaactggctgcgctttttcccgctgcgccacatccacattgtggacggcgaccgcctcatcagggaccccttccctgagatccaaaaggtcgagaggttcctaaagctgtcgccgcagatcaatgcttcgaacttctactttaacaaaaccaagggcttttactgcctgcgggacagcggccgggaccgctgcttacatgagtccaaaggccgggcgcacccccaagtcgatcccaaactactcaataaactgcacgaatattttcatgagccaaataagaagttcttcgagcttgttggcagaacatttgactggcac

SEQ ID NO: 46 - protein, 307 aa, Homo sapiens
MAALLLGAVLLVAQPQLVPSRPAELGQQELLRKAGTLQDDVRDGVAPNGSAQQLPQTIIIGVRKGGTRALLEMLSLHPDVAAAENEVHFFDWEEHYSHGLGWYLSQMPFSWPHQLTVEKTPAYFTSPKVPERVYSMNPSIRLLLILRDPSERVLSDYTQVFYNHMQKHKPYPSIEEFLVRDGRLNVDYKALNRSLYHVHMQNWLRFFPLRHIHIVDGDRLIRDPFPEIQKVERFLKLSPQINASNFYFNKTKGFYCLRDSGRDRCLHESKGRAHPQVDPKLLNKLHEYFHEPNKKFFELVGRTFDWH

(19) United States
(12) Patent Application Publication
(10) Pub. No.: US 2025/0154290 A1
(43) Pub. Date: May 15, 2025

(54) RECOMBINANT HEPARIN-LIKE SUBSTANCE AND METHOD FOR PRODUCING THE SAME

(71) Applicant: KYUSHU UNIVERSITY, NATIONAL UNIVERSITY CORPORATION, Fukuoka-shi, Fukuoka (JP)

(72) Inventors: Masamichi KAMIHIRA, Fukuoka-shi (JP); Yoshinori KAWABE, Fukuoka-shi (JP); Kyosuke AKIYAMA, Fukuoka-shi (JP)

(73) Assignee: KYUSHU UNIVERSITY, NATIONAL UNIVERSITY CORPORATION, Fukuoka-shi, Fukuoka (JP)

(21) Appl. No.: 18/838,797

(22) PCT Filed: Feb. 16, 2023
(86) PCT No.: PCT/JP2023/005371

§ 371 Date: Aug. 15, 2024

(30) Foreign Application Priority Data
Feb. 17, 2022 (JP) 2022-022594

(51) Int. Cl. C08B 37/00

(52) U.S. Cl. CPC C08B 37/0075

(57) ABSTRACT

To obtain a heparin-like substance with high biological activity (anticoagulation activity) as heparin. A method for producing a heparin-like substance is provided, which comprises the step of culturing an animal cell to which the followings are introduced to obtain a culture supernatant containing a heparin-like substance: a polynucleotide encoding glucosaminyl N-deacetylase/N-sulfotransferase (NDST2); a polynucleotide encoding heparan sulfate 3-O-sulfotransferase 1 (Hs3st1); a polynucleotide encoding the extracellular domain of syndecan (SDC); and a polynucleotide encoding 6-O-sulfotransferase.

### TECHNICAL FIELD

The present invention relates to a genetically engineering method for producing a heparin-like substance and a heparin-like substance produced thereby.

### BACKGROUND ART

Heparin is an essential drug used as an anticoagulant in medical settings such as dialysis and extracorporeal circulation. Heparin used in pharmaceuticals and other products is mainly extracted and purified from porcine small intestines or bovine lungs.

On the other hand, the production of heparin-like substances by genetic engineering techniques has been examined. Patent document 1 discloses the production of heparin based on gene transfer, and describes the use of non-human transgenic mammals modified to express heparin biosynthetic enzymes and core proteins. In addition, Patent document 1 lists genes relating to the sulfation pathway, including NDST1, NDST2 (Table 3) and Hs6st (Table 5-1) as heparin biosynthetic enzyme genes. This reference contains no experimental results of actual production of heparin-like substances by gene transfer, and their degree of sulfation and biological activity as heparin are unknown. Patent documents 2 to 4 describe porcine mast cells that produce heparin-type molecules and methods for producing heparin-type molecules using these cells. In particular, Patent document 2 describes the use of 3-O-sulfatase (3-OST) gene, and Patent document 3 describes the use of 3-OST and 6-OST genes. Further, Patent documents 2 to 4 describe analysis results about types and percentages of disaccharides in the obtained heparin-type molecules, as well as the biological activity of the obtained heparin-type molecules.

The inventors of the present invention have attempted to produce heparin-like substances using CHO cells that produce heparin-like substances. Then, they have found that heparin-like substances are secreted by recombinant CHO cells (CHO/NH-SDC), which are CHO cells introduced with genes involved in the production of heparin-like substances, (1) a gene encoding glucosaminyl N-deacetylase/N-sulfotransferase (hereinafter abbreviated as NDST2) and (2) a gene encoding heparan sulfate 3-O-sulfotransferase 1 (hereinafter abbreviated as Hs3st1), and further introduced with (3) a gene encoding the extracellular domain of syndecan (SDC), into the culture supernatant, and thus heparin-like substances can be efficiently produced (Patent document 5).

### PRIOR ART REFERENCES

### Patent Documents

Patent document 1: Japanese Patent Publication (Kohyo) No. 2017-503490
Patent document 2: Japanese Patent Publication (Kohyo) No. 2008-515417
Patent document 3: Japanese Patent Publication (Kohyo) No. 2006-522597
Patent document 4: Japanese Patent Publication (Kohyo) No. 2005-506092
Patent document 5: International Publication WO2021/066167

### SUMMARY OF THE INVENTION

### Problem to be Solved by the Invention

Since the heparin-like substance produced by the method of the inventors of the present invention (Patent document 5 mentioned above) is less sulfated than the heparins currently available on the market, and its biological activity as heparin (anticoagulant activity) is considerably lower than that of the commercial heparins, a solution to this problem is desired.

### Means for Solving the Problem

The inventors of the present invention investigated the possibility of secretory production of heparin-like sugar chains with biological activity equivalent to that of commercial heparins by further introducing sulfation pathway-related genes, such as the Hs6st3 gene, into the CHO cells mentioned above (CHO/NH-SDC). As a result, they found that the object can be achieved by introducing specific sulfation pathway-related genes, and accomplished the present invention.

The present invention provides the followings.

[1] A method for producing a heparin-like substance, which comprises the step of culturing an animal cell to which the followings are introduced to obtain a culture supernatant containing a heparin-like substance:
a polynucleotide encoding glucosaminyl N-deacetylase/N-sulfotransferase (NDST2);
a polynucleotide encoding heparan sulfate 3-O-sulfotransferase 1 (Hs3st1); —a polynucleotide encoding the extracellular domain of syndecan (SDC); and
a polynucleotide encoding 6-O-sulfotransferase.

[2] The production method according to 1, wherein the 6-O-sulfotransferase is any selected from the group consisting of heparan sulfate 6-O-sulfotransferase 1 (Hs6st1), heparan sulfate 6-O-sulfotransferase 2 (Hs6st2), and heparan sulfate 6-O-sulfotransferase 3 (Hs6st3).

[3] The production method according to 1 or 2, wherein the 6-O-sulfotransferase is Hs6st3.

[4] The production method according to any one of 1 to 3, wherein the animal cell is CHO cell.

[5] A recombinant heparin-like substance produced by the production method according to any one of 1 to 4, which has an anticoagulant factor Xa activity per mg of 200 IU/mg or higher.

[6] A recombinant CHO cell introduced with at least the followings:
a polynucleotide encoding NDST2;
a polynucleotide encoding Hs3st1;
a polynucleotide encoding the extracellular domain of SDC; and
a polynucleotide encoding 6-O-sulfotransferase.

[7] The cell according to 6, wherein the 6-O-sulfotransferase is any selected from the group consisting of Hs6st1, Hs6st2, and Hs6st3.

[8] The cell according to 6 or 7, wherein the 6-O-sulfotransferase is Hs6st3.

[9] The cell according to any one of 6 to 8, wherein the polynucleotide encoding 6-O-sulfotransferase is any of the followings:
(A) a polynucleotide consisting of the sequence of SEQ ID NO: 29 or 43;
(B) a polynucleotide that hybridizes with a polynucleotide consisting of a sequence complementary to the sequence of SEQ ID NO: 29 or 43 under stringent conditions and encodes a protein having a function to enhance the anticoagulant factor Xa activity of a heparin-like substance produced by a heparin-like substance-producing animal cell;
(C) a polynucleotide consisting of a sequence having an identity of 90% or higher to the sequence of SEQ ID NO: 29 or 43 and encoding a protein having a function to enhance the anticoagulant factor Xa activity of a heparin-like substance produced by a heparin-like substance-producing animal cell;
(D) a polynucleotide encoding a protein consisting of the amino acid sequence of SEQ ID NO: 30 or 44;
(E) a polynucleotide encoding a protein consisting of an amino acid sequence derived from the amino acid sequence of SEQ ID NO: 30 or 44 by substitution, deletion, insertion, and/or addition of multiple amino acids and having a function to enhance the anticoagulant factor Xa activity of a heparin-like substance produced by a heparin-like substance-producing animal cell; and
(F) a polynucleotide encoding a protein consisting of an amino acid sequence having an identity of 90% or higher to the amino acid sequence of SEQ ID NO: 30 or 44 and having a function to enhance the anticoagulant factor Xa activity of a heparin-like substance produced by a heparin-like substance-producing animal cell.

### BRIEF DESCRIPTION OF THE DRAWINGS

FIG. 1 Process for producing Hs6st3-expressing transposon vector.

FIG. 2 Sulfation Pathway.

FIG. 3 Anticoagulant factor Xa activity during transient expression. The anticoagulant factor Xa activity (anti-FXa activity) was measured in the culture supernatant of CHO cells introduced with sulfation pathway-related genes. The anti-FXa activity was significantly enhanced when, among the sulfation pathway-related genes, three enzymes involved in 6-O-sulfation, Hs6st3, Hs6st2, and Hs6st1, were introduced.

FIG. 4 Calculation of specific activity. The anticoagulant factor Xa activity (anti-FXa activity) per mg of substance (specific activity) was determined for culture supernatants of CHO cells introduced with sulfation pathway-related genes. Significantly enhanced specific activity was observed when any of the genes encoding the three enzymes Hs6st3, Hs6st2, and Hs6st1 was introduced. Expression of the Hs6st3 gene in CHO cells has not been confirmed until now.

FIG. 5 Enhancement of specific activity. The anticoagulant factor Xa activity (anti-FXa activity) per mg of sGAG (specific activity) was determined for the heparin-like substance produced by the Hs6st3-expressing cells (CHO/NH-SDC/Hs6st3). The specific activity was 95 IU/mg, a 9.7-fold enhancement over that obtained with conventional heparin-like substance-producing cells (CHO/NH-SDC). The specific activity of a commercial heparin is 248 IU/mg, and the recombinant CHO cell-produced heparin-like substance had about 38% of the activity.

FIG. 6 Evaluation of Hs6st3-expressing cell clones. Twenty-eight clones of Hs6st3-expressing cells (CHO/NH-SDC/Hs6st3) were obtained. A 1.5-to 9-fold enhancement was observed compared with the bulk cells.

FIG. 7 Evaluation of CHO/NH-SDC/Hs6st3 (#17). The anti-FXa activity (specific activity) of the heparin-like substance contained in the culture supernatant of the clone #17 of Hs6st3-expressing cells (CHO/NH-SDC/Hs6st3) is represented as a relative value to that of the commercial heparin.

FIG. 8 Evaluation of CHO/NH-SDC/Hs6st3 (#17). The anti-FIIa activity (specific activity) of the heparin-like substance contained in the culture supernatant of the clone #17 of Hs6st3-expressing cells (CHO/NH-SDC/Hs6st3) is represented as a relative value to that of the commercial heparin.

### MODES FOR CARRYING OUT THE INVENTION

### <Method for Producing Heparin-Like Substance>

The method for producing a heparin-like substance of the present invention is characterized by comprising the step of culturing an animal cell introduced with the followings to obtain a culture supernatant containing a heparin-like substance:
a polynucleotide encoding glucosaminyl N-deacetylase/N-sulfotransferase (NDST2);
a polynucleotide encoding heparan sulfate 3-O-sulfotransferase 1 (Hs3st1); —a polynucleotide encoding the extracellular domain of syndecan (SDC); and
polynucleotides encoding proteins involved in the respective steps of the sulfation pathway (transporters, enzymes, etc.).

### [Heparin-Like Substance]

For the present invention, the term heparin-like substance refers to heparin, heparan sulfate, or a mixture of these substances. Heparin can be said to be a highly sulfated version of heparan sulfate. Heparin and heparan sulfate are linear polysaccharides consisting of disaccharide repeating units, each of which consists of uronic acid (β-D-glucuronic acid or α-L-isuronic acid) and glucosamine (D-N-acetylglucosamine or D-glucosamine 2-N-sulfate), linked through α-1,4- or β-1,4-linkages. They include those in which the 2-position of uronic acid is O-sulfated and the 3- or 6-position of glucosamine is O-sulfated, and those in which the amino group at the 2-position of glucosamine is N-sulfated.

The heparin-like substance can be quantified by methods well known to those skilled in the art as sulfated glycosaminoglycan (sGAG). Further, the activity of heparin-like substance can be measured as the anticoagulant factor Xa (FXa) activity by methods well known to those skilled in the art. The activity can also be expressed as specific activity (activity per unit amount of protein). The heparin-like substance produced according to the present invention has a high sulfation level and thus enhanced anticoagulant activity. In general, low molecular weight heparins and synthetic heparins exhibit only the anti-FXa activity and do not exhibit the anticoagulant factor IIa (FIIa) activity. In contrast, the heparin-like substance produced according to the present invention can have both the anti-FXa and anti-FIIa activities. This indicates that the heparin-like substance produced according to the present invention may have a similar structure to commercial heparins.

### [Glucosaminyl N-Deacetylase/N-Sulfotransferase (NDST2)]

The animal cell used in the production method of the present invention is introduced with a polynucleotide encoding NDST2. NDST2 is a member of the N-deacetylase/N-sulfotransferase subfamily of the sulfotransferase 1 protein, and is an enzyme with two functions, functions to catalyze N-deacetylation and N-sulfation.

Preferred examples of the polynucleotide encoding NDST2 include any of those mentioned below:
(A2) a polynucleotide consisting of the sequence of SEQ ID NO: 21 or 39;
(B2) a polynucleotide that hybridizes with a polynucleotide consisting of a sequence complementary to the sequence of SEQ ID NO: 21 or 39 under stringent conditions and encodes a protein having the NDST2 activity;
(C2) a polynucleotide consisting of a sequence having an identity of 90% or higher to the sequence of SEQ ID NO: 21 or 39 and encoding a protein having the NDST2 activity;
(D2) a polynucleotide encoding a protein consisting of the amino acid sequence of SEQ ID NO: 22 or 40;
(E2) a polynucleotide encoding a protein consisting of an amino acid sequence derived from the amino acid sequence of SEQ ID NO: 22 or 40 by substitution, deletion, insertion, and/or addition of multiple amino acids and having the NDST2 activity; and (F2) a polynucleotide encoding a protein consisting of an amino acid sequence having an identity of 90% or higher to the amino acid sequence of SEQ ID NO: 22 or 40 and having the NDST2 activity.
   [Heparan sulfate 3-O-sulfotransferase 1 (Hs3st1)]

The animal cell used in the production method of the present invention is introduced with a polynucleotide encoding Hs3st1. Hs3st1 is a member of the heparan sulfate biosynthesis enzyme family, has both the heparan sulfate glucosaminyl 3-O-sulfotransferase activity and anticoagulant heparan sulfate conversion activity, and is the rate-limiting enzyme of the anticoagulant heparan synthesis.

Preferred examples of the polynucleotide encoding Hs3st1 include any of those mentioned below:
(A3) a polynucleotide consisting of the sequence of SEQ ID NO: 31, 41, or 45;
(B3) a polynucleotide that hybridizes with a polynucleotide consisting of a sequence complementary to the sequence of SEQ ID NO: 31, 41, or 45 under stringent conditions and encodes a protein having the Hs3st1 activity;
(C3) a polynucleotide consisting of a sequence having an identity of 90% or higher to the sequence of SEQ ID NO: 31, 41, or 45 and encoding a protein having the Hs3st1 activity;
(D3) a polynucleotide encoding a protein consisting of the amino acid sequence of SEQ ID NO: 32, 42, or 46;
(E3) a polynucleotide encoding a protein consisting of an amino acid sequence derived from the amino acid sequence of SEQ ID NO: 32, 42, or 46 by substitution, deletion, insertion, and/or addition of multiple amino acids and having the Hs3st1 activity; and
(F3) a polynucleotide encoding a protein consisting of an amino acid sequence having an identity of 90% or higher to the amino acid sequence of SEQ ID NO: 32, 42, or 46 and having the Hs3st1 activity.

### [Extracellular Domain of Syndecan (SDC)]

The animal cell used in the production method of the present invention is introduced with a polynucleotide encoding the extracellular domain of syndecan. Syndecans are members of the family of four types of cell surface proteoglycans with a retained plasma membrane domain and cytoplasmic domain. The structure of syndecan consists of the extracellular domain, transmembrane domain, and cytoplasmic domain. Of these, the extracellular domain contains a glycosaminoglycan-binding site.

Preferred examples of the polynucleotide encoding the extracellular domain of syndecan include any of those mentioned below:
(A4) a polynucleotide consisting of the sequence of SEQ ID NO: 37;
(B4) a polynucleotide that hybridizes with a polynucleotide consisting of a sequence complementary to the sequence of SEQ ID NO: 37 under stringent conditions and encodes a protein having a function to increase amount of a heparin-like substance in culture supernatant of a heparin-like substance-producing animal cell when it is introduced into the cell;
(C4) a polynucleotide consisting of a sequence having an identity of 90% or higher to the sequence of SEQ ID NO: 37 and encoding a protein having a function to increase amount of a heparin-like substance in culture supernatant of a heparin-like substance-producing animal cell when it is introduced into the cell;
(D4) a polynucleotide encoding a protein consisting of the amino acid sequence of SEQ ID NO: 38;
(E4) a polynucleotide encoding a protein consisting of an amino acid sequence derived from the amino acid sequence of SEQ ID NO: 38 by substitution, deletion, insertion, and/or addition of multiple amino acids and having a function to increase amount of a heparin-like substance in culture supernatant of a heparin-like substance-producing animal cell when it is introduced into the cell; and
(F4) a polynucleotide encoding a protein consisting of an amino acid sequence having an identity of 90% or higher to the amino acid sequence of SEQ ID NO: 38 and having a function to increase amount of a heparin-like substance in culture supernatant of a heparin-like substance-producing animal cell when it is introduced into the cell.

Concerning the polynucleotide encoding the extracellular domain of syndecan, the expression of having a function to increase amount of a heparin-like substance in culture supernatant means that when the polynucleotide in question is introduced into a heparin-like substance-producing animal cell to which any polynucleotide encoding the extracellular domain of syndecan has not been introduced, amount of a heparin-like substance in culture supernatant is increased compared with that observed before the introduction. Examples of the heparin-like substance-producing animal cell referred to here include a CHO cell to which a polynucleotide encoding NDST2 and a polynucleotide encoding Hs3st1 have been introduced. A more specific example is the CHO-S/NH cell (see Patent document 5).

### [Proteins Involved in Respective Steps of Sulfation Pathway (Transporters, Enzymes, Etc.)]

The animal cell used in the production method of the present invention is introduced with polynucleotides encoding proteins involved in the respective steps of the sulfation pathway (transporters, enzymes, etc.). The polynucleotides encoding proteins involved in the respective steps of the sulfation pathway include the followings:
a gene encoding a transporter of sulfate ions into cell (e.g., SLC26A1, SLC26A2, SLC13A1, and SLC13A4),
a gene encoding an enzyme that synthesize PAPS from sulfate ions (e.g., SLC26A1, SLC26A2, SLC13A1, and SLC13A4),
a gene encoding a transporter of PAPS to the Golgi apparatus (e.g., PAPSS1 and PAPSS2),
a gene encoding a glycan conversion enzyme (e.g., Glce),
a gene encoding a glucosaminyl N-deacetylase/N-sulfotransferase (e.g., NDST1 and NDST2),
a gene encoding a 2-O-sulfotransferase (e.g., Hs2st),
a gene encoding a 6-O-sulfotransferase (e.g., Hs6st1, Hs6st2, and Hs6st3), and
a gene encoding a 3-O-sulfotransferase (e.g., Hs3st1 and Hs3st5)

According to the studies of the inventors of the present invention, among the genes encoding the proteins involved in the respective steps of the sulfation pathway, it is preferable to introduce a 6-O-sulfotransferase gene, and it is more preferable to introduce the Hs6st3 gene. This is because introduction of such a gene increases the sulfation level of the heparin-like substance produced by a heparin-like substance-producing animal cell and enhances the anticoagulant factor Xa activity of the same.

Preferred examples of the Hs6st3 gene, i.e., polynucleotide encoding Hs6st3, include any of the followings:
(A) a polynucleotide consisting of the sequence of SEQ ID NO: 29 or 43;
(B) a polynucleotide that hybridizes with a polynucleotide consisting of a sequence complementary to the sequence of SEQ ID NO: 29 or 43 under stringent conditions and encodes a protein having a function to enhance the anticoagulant factor Xa activity of a heparin-like substance produced by a heparin-like substance-producing animal cell;
(C) a polynucleotide consisting of a sequence having an identity of 90% or higher to the sequence of SEQ ID NO: 29 or 43 and encoding a protein having a function to enhance the anticoagulant factor Xa activity of a heparin-like substance produced by a heparin-like substance-producing animal cell;
(D) a polynucleotide encoding a protein consisting of the amino acid sequence of SEQ ID NO: 30 or 44;
(E) a polynucleotide encoding a protein consisting of an amino acid sequence derived from the amino acid sequence of SEQ ID NO: 30 or 44 by substitution, deletion, insertion, and/or addition of multiple amino acids and having a function to enhance the anticoagulant factor Xa activity of a heparin-like substance produced by a heparin-like substance-producing animal cell; and
(F) a polynucleotide encoding a protein consisting of an amino acid sequence having an identity of 90% or higher to the amino acid sequence of SEQ ID NO: 30 or 44 and having a function to enhance the anticoagulant factor Xa activity of a heparin-like substance produced by a heparin-like substance-producing animal cell.

Preferred examples of the Hs6st1 gene, i.e., polynucleotide encoding Hs6st1, include any of the followings:
(A5) a polynucleotide consisting of the sequence of SEQ ID NO: 25;
(B5) a polynucleotide that hybridizes with a polynucleotide consisting of a sequence complementary to the sequence of SEQ ID NO: 25 under stringent conditions and encodes a protein having a function to enhance the anticoagulant factor Xa activity of a heparin-like substance produced by a heparin-like substance-producing animal cell;
(C5) a polynucleotide consisting of a sequence having an identity of 90% or higher to the sequence of SEQ ID NO: 25 and encoding a protein having a function to enhance the anticoagulant factor Xa activity of a heparin-like substance produced by a heparin-like substance-producing animal cell;
(D5) a polynucleotide encoding a protein consisting of the amino acid sequence of SEQ ID NO: 26;
(E5) a polynucleotide encoding a protein consisting of an amino acid sequence derived from the amino acid sequence of SEQ ID NO: 26 by substitution, deletion, insertion, and/or addition of multiple amino acids and having a function to enhance the anticoagulant factor Xa activity of a heparin-like substance produced by a heparin-like substance-producing animal cell; and
(F5) a polynucleotide encoding a protein consisting of an amino acid sequence having an identity of 90% or higher to the amino acid sequence of SEQ ID NO: 26 and having a function to enhance the anticoagulant factor Xa activity of a heparin-like substance produced by a heparin-like substance-producing animal cell.

Preferred examples of the Hs6st2 genes, i.e. polynucleotide encoding Hs6st2, include any of the followings:
(A6) a polynucleotide consisting of the sequence of SEQ ID NO: 27;
(B6) a polynucleotide that hybridizes with a polynucleotide consisting of a sequence complementary to the sequence of SEQ ID NO: 27 under stringent conditions and encodes a protein having a function to enhance the anticoagulant factor Xa activity of a heparin-like substance produced by a heparin-like substance-producing animal cell;
(C6) a polynucleotide consisting of a sequence having an identity of 90% or higher to the sequence of SEQ ID NO: 27 and encoding a protein having a function to enhance the anticoagulant factor Xa activity of a heparin-like substance produced by a heparin-like substance-producing animal cell;
(D6) a polynucleotide encoding a protein consisting of the amino acid sequence of SEQ ID NO: 28;
(E6) a polynucleotide encoding a protein consisting of an amino acid sequence derived from the amino acid sequence of SEQ ID NO: 28 by substitution, deletion, insertion, and/or addition of multiple amino acids and having a function to enhance the anticoagulant factor Xa activity of a heparin-like substance produced by a heparin-like substance-producing animal cell; and
(F6) a polynucleotide encoding a protein consisting of an amino acid sequence having an identity of 90% or higher to the amino acid sequence of SEQ ID NO: 28 and having a function to enhance the anticoagulant factor Xa activity of a heparin-like substance produced by a heparin-like substance-producing animal cell.

The expression that a polynucleotide encoding 6-O-sulfotransferase has a function to enhance the anticoagulant factor Xa activity of a heparin-like substance produced by a heparin-like substance-producing animal cell means that when the polynucleotide is introduced into a heparin-like substance-producing animal cell to which any polynucleotide encoding 6-O-sulfotransferase has not been introduced, the anticoagulant factor Xa activity of the heparin-like substance produced by the heparin-like substance-producing animal cell is enhanced compared with that observed before the introduction. Examples of the heparin-like substance-producing animal cell referred to here include a CHO cell into which polynucleotides encoding NDST2, Hs3st1, and the extracellular domain of SDC have been introduced. A more specific example is the CHO-S/NH-SDC cell (see Patent document 5).

[Anticoagulant with High Specific Activity]

By the production method of the present invention, a heparin-like substance having an anticoagulant factor Xa activity per mg (specific activity) of 8 IU/mg or higher can be obtained. According to preferred embodiments, the specific activity can be 10 IU/mg or higher, 12 IU/mg or higher, 30 IU/mg or higher, 60 IU/mg or higher, or 90 IU/mg or higher. The term IU (International Unit) used for heparin-like substances in the present invention is used to mean the IU of the anticoagulant factor Xa activity (IU anti-Xa activity), unless especially stated. The IU can be defined according to the international standard (IS) recommended in WHO/BS/2012.2207, and IU value of a subject substance can be determined by using IS.

In the present invention, an anticoagulant factor Xa activity (specific activity) numerically indicated is a value calculated by dividing a value measured as heparin concentration by a method based on the following measurement principle by a value measured as total glucosaminoglycan concentration by a method based on the following measurement principle, unless especially stated.

### Principle of Heparin Concentration Measurement

When antithrombin III is added to a sample to form a heparin-antithrombin III complex, and a certain excess amount of the anticoagulant factor Xa is added to the complex and then allowed to react, the heparin-antithrombin III complex binds to anticoagulant factor Xa in a proportional manner, forming an inactive heparin-antithrombin III/anticoagulant factor Xa complex. If the substrate (N-benzoyl-L-isoleucyl-L-glutamyl (γ-OR)-glycyl-L-arginyl-p-nitroanilide hydrochloride) is added to the complex, p-nitroaniline is released in an amount corresponding to the residual anticoagulant factor Xa activity. Since this residual anticoagulant factor Xa activity reflects the heparin concentration in the sample, the heparin concentration in the sample can be determined by colorimetrically quantifying the released p-nitroaniline at 405 nm.

Measurement based on this principle can be performed by using a commercially available heparin assay kit, for example, Testzym Heparin S #30564000 (Sekisui Medical). In this case, reagents sold as standards, e.g., heparin sodium (product codes 081-00131, 085-00134, 081-00136, and 087-00133, Fujifilm Wako Pure Chemicals) can be used.

### Principle of Glucosaminoglycan Measurement

Blyscan Dye (1,9-dimethyl-methylene blue), which specifically binds to sulfated glycans, is added to a sample to pelletize soluble sulfated proteoglycans and sulfated glycosaminoglycans contained in the sample. The dye can be eluted from the pellet with a reagent and colorimetrically quantified at 656 nm to determine the concentration of the total glucosaminoglycans contained in the sample.

Measurement based on this principle can be performed by using a commercially available glycosaminoglycan assay kit, such as the Blyscan Glycosaminoglycan Assay Kit (product code B1000, Biocolor).

As described below, by using cells selected from the recombinant CHO cell population obtained according to the present invention, an anticoagulant showing a specific activity equivalent to or higher than that of the commercial heparin (specific activity 248 IU/mg), for example, 250 IU/mg, 300 IU/mg, or 400 IU/mg, can be produced. Recombinant heparin-like substances with such a high specific activity are novel. Therefore, the present invention provides a recombinant heparin-like substance having a specific activity of 200 IU/mg or higher, preferably 230 to 270 IU/mg, more preferably 240 to 260 IU/mg.

### [Heparin-Like Substance-Producing Animal Cell]

Concerning the present invention, the term heparin-like substance-producing animal cell means an animal cell having the proteins necessary for the heparin biosynthesis and capable of producing a heparin-like substance, unless especially noted.

Such a heparin-like substance-producing animal cell can be obtained by introducing polynucleotides required for the heparin biosynthesis into an animal cell. For example, if the animal cell is a CHO cell, a heparin-like substance-producing CHO cell can be obtained by introducing at least one, preferably both, of the polynucleotides encoding NDST2 and Hs3st1, which are required for the heparin biosynthesis and are not expressed in CHO cells (Bail J Y et al., Metab Eng14: 81-90, 2012).

Examples of the animal cell used in the present invention include, for example, Chinese hamster ovary cells [CHO cells, Journal of Experimental Medicine, 108, 945 (1958); Proc. Natl. Acad. Sci. USA, 60, 1275 (1968); Genetics, 55, 513 (1968); Chromosoma, 41, 129 (1973); Methods in Cell Science, 18, 115 (1996); Radiation Research, 148, 260 (1997); Proc. Natl. Acad. Sci., USA, 77, 4216 (1980); Proc. Natl. Acad. Sci., USA, 60, 1275 (1968); Cell, 6, 121 (1975); and Molecular Cell Genetics, Appendix I, II (pp. 883-900)], CHO cells deficient in the dihydrofolate reductase gene [CHO/DG44 cells, Proc. Natl. Acad. Sci. USA, 77, 4216 (1980)], CHO-K1 (ATCC CCL-61), DUKXB11 (ATCC CCL-9096), Pro-5 (ATCC CCL-1781), CHO-S (Life Technologies, Cat #11619), Pro-3, human umbilical vein endothelial cells (HUVEC), human umbilical artery endothelial cells (HUAEC), human lung microvascular endothelial cells (HLMVEC), human aortic endothelial cells (HAoEC), human coronary artery endothelial cells (HCAEC), human pulmonary arterial endothelial cells (HPAEC), human fetal kidney (HEK) cells, rat myeloma cells YB2/3HL.P2.G11.16Ag.20 (also referred to as YB2/0), monkey COS cells, NSO mouse myeloma cells, mouse myeloma cells SP2/0-Ag14, Syrian hamster cells BHK or HBT5637 (Japanese Patent Publication (Kokai) No. 63-000299), and so forth. Among these, CHO cells, CHO/DG44 cells and CHO-K1 (ATCC CCL-61) are preferred, and CHO cells are more preferred, in terms of production efficiency.

### [Polynucleotides and Proteins]

The polynucleotides and proteins referred to in the present invention may be derived from humans or mammalian animals other than humans, unless especially noted. Examples of the mammalian animals other than humans include pigs, mice, rats, and hamsters.

Concerning the present invention, the hybridization conditions for the definition that a polynucleotide that hybridizes under stringent conditions can be appropriately chosen depending on the polynucleotide to be obtained according to the descriptions of Molecular Cloning. A Laboratory Manual. 4th ed. (Sambrook et al., Cold Spring Harbor Laboratory Press) and Hybridization of Nucleic Acid Immobilization on Solid Supports (ANALYTICAL BIOCHEMISTRY 138, 267-284 (1984)) for any polynucleotides, unless especially noted. For example, when a DNA having an identity of 50% or higher is to be obtained, there can be used such hybridization conditions that hybridization is performed at 40° C. in the presence of 6×SSC solution (the composition of 1×SSC solution consists of 150 mM sodium chloride and 15 mM sodium citrate) and 5% formamide, and then the filter is washed at 49° C. with 4×SSC solution. To obtain a DNA having an identity of 85% or higher, there can be used such hybridization conditions that hybridization is performed at 40° C. in the presence of 2×SSC solution and 50% formamide, and then the filter is washed at 57° C. with 0.1×SSC solution. To obtain a DNA having an identity of 90% or higher, there can be used such hybridization conditions that hybridization is performed at 45° C. in the presence of 2×SSC solution and 50% formamide, and then the filter is washed at 62° C. with 0.1×SSC solution.

Concerning the present invention, in the definition of an amino acid sequence derived by substitution, deletion, insertion, and/or addition of one or more amino acids, the number of amino acids replaced or the like is not particularly limited for any proteins as long as the protein consisting of the amino acid sequence has the desired function, unless especially specified, but may be about 1 to 250, 1 to 200, 1 to 150, 1 to 100, 1 to 50, 1 to 40, 1 to 30, 1 to 20, 1 to 15, 1 to 9 or 1 to 4, or in the case of substitutions of amino acids of similar nature, a further larger number of amino acids may be replaced or the like. Means for preparing polynucleotides or proteins relating to such amino acid sequences are well known to those skilled in the art.

Concerning the present invention, the term identity used for nucleotide sequences (also referred to as base sequences) or amino acid sequences means the percentage of the number of matching nucleotides or amino acids shared between two sequences aligned in an optimal manner, unless especially stated. That is, the identity can be calculated in accordance with the equation: Identity=(Number of matched positions/Total number of positions)×100, and can be calculated by using commercially available algorithms. Such algorithms are incorporated into the NBLAST and XBLAST programs described in Altschul et al. J. Mol. Biol. 215 (1990) 403-410. In more detail, the search and analysis for sequence identity can be performed by using algorithms or programs known to those skilled in the art (e.g., BLASTN, BLASTP, BLASTX, and ClustalW). When a program is used, the parameters can be appropriately set by those skilled in the art, or the default parameters of each program can be used. The specific methods of these analyses are also well known to those skilled in the art. The genetic information processing software GENETIX (registered trademark, Genetix) may also be used to calculate the identity. If a sequence for which % identity is sought has an additional sequence at the end, such as a tag sequence, that is not present in the sequence to be compared, the additional sequence portion is not included in the calculation of the % identity.

Concerning the present invention, the expression that a nucleotide or amino acid sequence has an “identity” means that the nucleotide or amino acid sequence has an identity of at least 50%, for example, 60% or higher, or 70% or higher, preferably 80% or higher, more preferably 85% or higher, further preferably 90% or higher, still further preferably 95% or higher, still further preferably 97.5% or higher, still further preferably 99% or higher, in any case, unless especially noted.

The polynucleotides or genes and proteins or enzymes used in the present invention can be prepared by those skilled in the art using conventional techniques.

The default parameters of the programs are as follows: G (cost to open gap) is 5 for nucleotide sequences and 11 for amino acid sequences; −E (cost to extend gap) is 2 for nucleotide sequences and 1 for amino acid sequences; −q (penalty for nucleotide mismatch) is −3; −r (reward for nucleotide match) is 1; −e (expect value) is 10; −W (wordsize) is 11 residues for nucleotide sequences and 3 residues for amino acid sequences, −y [Dropoff (X) for blast extensions in bits] is 20 for blastn and 7 for programs other than blastn, −X (X dropoff value for gapped alignment in bits) is 15, and −Z (final X dropoff value for gapped alignment in bits) is 50 for blastn and 25 for programs other than blastn (https://blast.ncbi.nlm.nih.gov/Blast.cgi?PAGE_TYPE=BlastSearch).

Polypeptides having an amino acid sequence derived from the desired amino acid sequence by substitution, deletion, insertion, and/or addition of one to several amino acids can be obtained by introducing a site-directed mutation into, for example, DNA encoding a polypeptide containing any of the amino acid sequences of SEQ ID NOS: 1 to 3 by using the site-directed mutagenesis method [Molecular Cloning, A Laboratory Manual, Second Edition, Cold Spring Harbor Laboratory Press (1989); Current Protocols in Molecular Biology, John Wiley & Sons (1987-1997); Nucleic Acids Research, 10, 6487 (1982); Proc. Natl. Acad. Sci. USA, 79, 6409 (1982); Gene, 34, 315 (1985); Nucleic Acids Research, 13, 4431 (1985); and Proc. Natl. Acad. Sci. USA, 82, 488 (1985)], or the like.

### <Recombinant Cell>

The present invention provides a recombinant CHO cell into which at least the followings have been introduced:
a polynucleotide encoding NDST2;
a polynucleotide encoding Hs3st1;
a polynucleotide encoding the extracellular domain of syndecan (SDC); and
a polynucleotide encoding 6-O-sulfotransferase.

Such an animal cell can be obtained by introducing a recombinant vector containing a predetermined polynucleotide into an animal cell that will serve as a host cell. The above explanation can be directly applied to each polynucleotide.

Any recombinant vector can be used as long as it is capable of autonomous replication or incorporation into the chromosome in the host cell to be used and contains an appropriate promoter at such a position that DNA encoding a polypeptide can be transcribed.

Although a transcription termination sequence is not necessarily required for the recombinant vector, it is preferable to place a transcription termination sequence immediately downstream of the structural gene. In addition, the recombinant vector may contain a gene that controls the promoter.

As the recombinant vector, it is preferable to use a plasmid with the Kozak sequence, which is a ribosome-binding sequence, appropriately placed around the start codon.

In the nucleotide sequences of DNA, nucleotides can be replaced so as to obtain codons optimal for expression in the host, thereby increasing the production rates of the target NDST2 and Hs3st1.

Any vector that can function in animal cells can be used for the recombinant vector, and examples include, for example, pcDNA I, pcDM8 (Funakoshi), pAGE107 [Japanese Patent Publication (Kokai) No. Hei 03-22979; and Cytotechnology, 3, 133 (1990)], pAS3-3 (Japanese Patent Publication (Kokai) No. Hei 02-227075), pcDM8 [Nature, 329, 840 (1987)], pcDNA I/Amp (Invitrogen), pcDNA3.1 (Invitrogen), pREP4 (Invitrogen), pAGE103 [J. Biochemistry 101, 1307 (1987)], pAGE210, pME18SFL3, pKANTEX93 (WO97/10354), N5KG1val (U.S. Pat. No. 6,001,358), INPEP4 (Biogen-IDEC), transposon vector (WO2010/143698), and so forth.

Any promoter that can function in animal cells can be used, and examples include, for example, the promoter of the immediate early (IE) gene of cytomegalovirus (CMV), early promoter of SV40, promoter of retrovirus, metallothionein promoter, heat shock promoter, SRa promoter, and promoter or enhancer of Moloney murine leukemia virus. The enhancer of the IE gene of human CMV may also be used with a promoter.

The recombinant vector may contain a selection marker. The selection marker is a gene that allows selection of cells containing the gene. Selection can be positive selection or negative selection. Positive selection refers to a process in which positive selection occurs to select cells containing the selection marker. Drug resistance is an example of the positive selection marker, and cells containing the marker survive in the drug-containing culture medium, while cells without the marker die. Examples of the selection marker include drug resistance genes such as neo, which confers G418 resistance; hygr, which confers hygromycin resistance; and puro, which confers puromycin resistance. Other examples of the positive selection marker gene include genes that allow identification of or screening for cells containing a marker. Examples of such genes include, among others, fluorescent protein (GFP and GFP-like chromophores, luciferase etc.) genes, lacZ gene, alkaline phosphatase gene, and surface markers such as CD8. Negative selection refers to a process in which cells containing a negative selection marker are killed by exposure to an appropriate negative selection agent. For example, cells containing the herpes simplex virus thymidine kinase (HSV-tk) gene [Wigler et al, Cell 11:223 (1977)] are sensitive to the drug ganciclovir (GANC). Similarly, the gpt gene makes cells susceptible to 6-thioxanthin.

Any method for introducing DNA into animal cells can be used to introduce a recombinant vector into host cells. Examples include, for example, the electroporation method [Cytotechnology, 3, 133 (1990)], calcium phosphate method (Japanese Patent Publication (Kokai) No. Hei 02-227075), and lipofection method [Proc. Natl. Acad. Sci. USA, 84, 7413 (1987)], and so forth.

Recombinant cells are preferably cultured under such conditions that the production of a heparin-like substance is promoted. Specifically, recombinant cells are preferably cultured, for example, in a medium that allows heparin-like substance production by the recombinant cells and promotes secretion of a heparin-like substance from the recombinant cells into the culture supernatant.

The medium for culturing the recombinant cells preferably contains carbon, nitrogen, oxygen and other nutrients, growth factors, buffering agents, cofactors and any other substances sufficient for at least maintaining cell viability and allowing expression of a heparin-like substance. In an embodiment where the gene encoding the heparin-like substance is under the control of an inducible promoter or contains an inducible promoter, the medium may also contain an inducer.

Examples of the medium include, for example, RPMI or DMEM supplemented with 10% fetal calf serum (FCS), as well as tissue culture media supplemented with antimicrobial agents, growth factors, and other factors such as cytokines (e.g., Cell Biology (Third Edition) A Laboratory Handbook, vol. 1, 2006, Elsevier Inc.). Specific examples include, for example, media preparations known to those skilled in the art, such as RPMI, IMDM, DMEM, DMEM/F12, and serum-free or low-serum EMEM. These media may contain antibiotics, additional nutritional supplements such as lipids, transferrin, insulin, and amino acids, and cofactors as needed.

From the viewpoint of promoting the production of the heparin-like substance, the medium preferably contains at least one selected from glucose, sulfate and phosphoric acid. The concentration of glucose in the medium is usually preferably 5 to 75 mM, more preferably 10 to 60 mM, still more preferably 15 to 35 mM. The concentration of sulfate in the medium is usually preferably 0.5 to 50 mM, more preferably 10 to 50 mM, still more preferably 30 to 50 mM. The concentration of phosphate in the medium is usually preferably 0.5 to 50 mM, more preferably 1 to 50 mM, still more preferably 10 to 50 mM.

By allowing generation and accumulation of the heparin-like substance in the culture supernatant and collecting it from the culture supernatant, the heparin-like substance can be produced. The method for culturing recombinant cells in the culture medium may be a usual method. The cultures is usually performed for 1 to 7 days under such conditions as pH 6 to 8, 30 to 40° C., and the presence of 5% CO2.

Secretory production of the heparin-like substance from the recombinant cells can be confirmed by subjecting the culture supernatant to an enzyme treatment by adding an enzyme solution containing heparin lyase I, II, or III, and quantifying the reaction product by HPLC for unsaturated disaccharide analysis. Secretory production of the heparin-like substance from the recombinant cells can also be confirmed by measuring the amount of sGAG in the culture supernatant.

The recombinant cell of the present invention secretes a proteoglycan, which is a glycoprotein containing a core protein binding to the heparin-like substance, GAG, in the culture supernatant. Isolation of the protein from the culture supernatant is performed by a method conventionally known in the field. For example, a tag that facilitates the isolation of the heparin-like substance, such as affinity tag, may be used. Examples of the tag include, for example, polyhistidine (His6 tag), nickel matrix, chitin-binding protein (CBP), maltose-binding protein (MBP), glutathione-S-transferase (GST), FLAG tag, and epitope tag.

Isolation of the heparin-like substance from the core protein is performed by a method known in the art. Examples include enzymatic digestion with heparinase and treatment with sodium hydroxide or alkaline borohydride.

The resulting heparin-like substance may contain repeating structures consisting of disaccharide units and having various lengths. The heparin-like substance preferably contains any of UA-GlcNAc (6S), UA (2S)-GlcNAc, UA (2S)-GlcNAc (6S), UA-GlcNS, UA-GlcNS (6S), UA (2S)-GlcNS, and UA (2S)-GlcNS (6S), which may be present in any order in the heparin-like substance.

In the above description, UA is an uronic acid residue (i.e., glucuronic acid or isuronic acid), Ac is acetyl, GlcNAc is N-acetylglucosamine, GlcNS is glucosamine-N-sulfate, 2S is 2-O-sulfate, and 6S is 6-O-sulfate.

### <Pharmaceutical Compositions>

The present invention provides a pharmaceutical composition containing a heparin-like substance or a fragment thereof produced by the method described herein. The heparin-like substance or fragment thereof contained in the pharmaceutical composition may be bound to a core protein. The pharmaceutical composition may contain another therapeutic agent. The pharmaceutical composition may also be formulated by using, for example, conventionally used vehicles or diluents, as well as a pharmaceutical additive (e.g., excipient, binder, and preservative) of a type suitable for the desired administration method.

Examples of the form of the pharmaceutical composition include a sterile aqueous preparation for injection. Such a sterile aqueous preparation for injection can be prepared according to known techniques using an appropriate dispersant or wetting agent and suspending agent. The sterile aqueous preparation for injection may be a sterile solution or suspension for injection in a non-toxic parenterally acceptable diluent or solvent, such as, for example, a solution in 1,3-butanediol. Examples of the acceptable vehicle and solvent that can be used include water, Ringer's solution, and isotonic saline.

The dosage form of the pharmaceutical composition is not particularly limited, and a wide variety of dosage forms can be used. As for the dosage form for administering the pharmaceutical composition, the composition can be administered in the form of, for example, tablet, capsule, sachet, troche, pill, powder, granule, elixir, tincture, solution, suspension, elixir, syrup, ointment, cream, or the like, or intravenously administered. Examples of the dosage form also include injection, paste, emulsion and solution. Examples of the administration scheme also include, for example, transdermal administration using patch mechanism or ointment. Any of these dosage forms may be prepared as sustained release and/or extended release preparations.

Examples of pharmaceutically acceptable carrier include, but are not limited to, vehicles, adjuvants, surfactants, suspending agents, emulsifiers, inert fillers, diluents, excipients, wetting agents, binders, lubricants, buffering agents, disintegrants and carriers. Typically, such pharmaceutically acceptable carriers are chemically inert to the active compound and have no adverse side effects or toxicity under the conditions for use. The natures of the pharmaceutically acceptable carriers may vary depending on the specific dosage form used and other characteristics of the composition.

### <Treatment Method and Prevention Method>

The present invention provides a method for treating or preventing blood coagulation or a condition relating to or caused by blood coagulation in a subject in need thereof, which comprises administering a heparin-like substance or a fragment thereof produced by the method of the present invention. In this aspect, the heparin-like substance or a fragment thereof may be bound to a core protein. Examples of blood coagulation or condition relating to or caused by blood coagulation include, for example, acute coronary syndrome, atrial fibrillation, deep vein thrombosis, and pulmonary embolism.

The subject is a mammal. Examples of the mammal include, for example, humans, primates, domestic animals (e.g., sheep, cattle, horse, donkey, and pig), companion animals (e.g., dog and cat), laboratory test animals (e.g., mouse, rabbit, rat, guinea pig, and hamster), and captured wild animals (e.g., fox and deer). The mammal is typically human or primate, more typically human.

The dose and administration time are preferably such a dose and administration time that therapeutic benefits are provided in the treatment, prevention or management of blood coagulation or condition relating to blood coagulation. Specific effective dose and administration time may vary depending on such factors as the subject's condition, medical history, physique, weight, and age.

Examples of the present invention will be given below, but the present invention is not limited to the following examples.

### EXAMPLES

### Experimental Materials and Methods

### 1. CHO Cell Culture

### (1) Composition of Culture Medium

FreeStyle CHO Expression Medium (Cat. No. 12651014, Invitrogen) was used as the medium for culturing CHO-S cells (Cat. No. R80007, Invitrogen) and CHO/NH-SDC cells. Penicillin (Cat. No. 021-07732, Wako) and streptomycin (Cat. No. 194-08512, Wako) were added as antibiotics. L-Glutamine (Cat. No. 074-00522, Wako) was also added to the medium at a final concentration of 8 mM. The cells were inoculated in 5 mL of the medium in T-25 flasks for floating cells (Cat. No. 690195, Griener) and cultured at 37° C. under a humidified atmosphere in a 5% CO2 incubator. As the inventors had experienced, the wild-type CHO-S cells reached 100% confluence at a density of approximately 1.5×10⁷ cells/flask, and in the case of subculture, when the cells were cultured at a density of 0.3×10⁷ cells/flask (about ⅕), they reached confluence after 2 days. The CHO/NH-SDC cells reached 100% confluency at a density of approximately 1.2×10⁷ cells/flask, and in the case of subculture, when the cells were subcultured at a density of 0.3×10⁷ cells per flask (about ¼), they reached the confluency after 3 days. As described below, in the experiments such as serum-free acclimation, the growth state was maintained by increasing the seeding density.

For antibiotics and glutamine, concentrated solutions were prepared and added according to the following procedure. For antibiotics, 69.9 mg of penicillin and 100 mg of streptomycin were dissolved in 10 mL of 0.85% aqueous NaCl, and each solution was filtered through a 0.45 μm syringe filter for sterilization in a laminar flow cabinet to prepare a 100× stock solution. For glutamine, 1.169 g of L-glutamine was dissolved in 10 mL of 0.85% aqueous NaCl, and the solution was filtered through a 0.45 μm syringe filter for sterilization in a laminar flow cabinet to prepare a 200 mM stock solution.

When the cells were cultured in an adherent state, the medium was prepared as follows. The basal medium F12 (10.6 g/L, Cat. No. N6760, Sigma) containing 10% (v/v) fetal bovine serum (FBS) (Cat. No. FB-1280/500, Lot. No. 015BS281, Biosera) was used. As antibiotics, 70 mg/L penicillin and 100 mg/L streptomycin were used, and 1.176 g/L sodium hydrogencarbonate (Cat. No. 191-01305, Wako) was also used. The cells were cultured on normal dishes (Cat. No. 130182, Thermo) at 37° C. in a 5% CO2 incubator.

### (2) Cell Adhesion and Serum-Free Acclimation

The CHO-S cells and CHO-S/NH-SDC cells established in the laboratory of the inventors of the present invention (see Patent document 5) were originally floating cultured in the serum-free medium, FreeStyle CHO Expression Medium. However, it was necessary to culture the cells by adhesion culture when performing gene transfer by lipofection or cloning by the limiting dilution method. Therefore, F12+10% FBS medium was used for adhesion. When the adhesion culture was no longer necessary, serum-free acclimation was performed. For serum-free acclimation, serum-containing medium containing 5%, 1%, 0.5%, or 0.1% serum was prepared by mixing F12+10% FBS and FreeStyle CHO-S Expression Medium in an appropriate ratio, and used. First, the cells cultured in F12+10% FBS medium were subcultured 2 or 3 times in the medium containing 5% serum. The same process was then repeated in turn to reduce the serum concentration to complete the serum-free acclimation. During the serum-free acclimation, the subculture was performed with efforts to maintain a higher cell density (0.5×10⁶ cells/flask) compared with normal culture.

### 2. Method for Evaluating Cell Product

### (1) Measurement of Anticoagulant Factor Xa Activity of Culture Supernatant

Anticoagulant activity of the heparin-like substance contained in the culture supernatant of the cells was evaluated by measuring anticoagulant factor Xa activity (anti-FXa activity). Established cell clones or cells of each cell line were inoculated in FreeStyle CHO-S Expression Medium at a cell density of 0.6×10⁶ cells/well in a volume of 2 mL/well using a 6-well plate (Cat. No. 130184, Thermo) on day 0. Culture was performed without medium change and subculture, and the culture supernatant was collected on day 3. The undiluted solution of the culture supernatant was used as a sample.

The measurement was performed by using BIOPHEN HEPARIN ANTI-Xa (2 stages) (Cat. No. 221005, HBM), if the activity of the sample was low, or Testzym Heparin S assay kit (Cat. No. 30564000, Sekisui Medical), if high activity was expected. The operation was performed according to the manual of each kit.

The principles of the measurement with the kits are the same. When antithrombin III is added to a sample containing heparin to form a heparin-antithrombin III complex, and the complex is then reacted with a certain excess amount of factor Xa (FXa), the heparin-antithrombin III complex binds to FXa in a proportional manner, forming an inactive heparin-antithrombin III-FXa complex. If an FXa-specific chromogenic substrate is added to this reaction mixture, a dye corresponding to the residual FXa activity is released. Since the residual activity of FXa reflects the anticoagulant activity of heparin contained in the sample, the dye released from the chromogenic substrate is colorimetrically quantified at 405 nm. Brief description of the operation for using each kit is as follows.

### (a) BIOPHEN HEPARIN ANTI-Xa (2 Stages)

When BIOPHEN HEPARIN ANTI-Xa (2 stages) is utilized, BIOPHEN UFC Calibrator (Cat. No. 222301-RUO, HBM), which contains samples of known activity values, was used for the preparation of a calibration curve. The five kinds of powder samples contained in the BIOPHEN UFC Calibrator kit were each dissolved by adding 1 mL of sterile water. These solutions were diluted 15-fold by adding Tris NaCl EDTA PEG buffer-pH 8.40 (Cat. No. AR030, HBM) to prepare five kinds of standard solutions. Antithrombin III powder (R1), FXa powder (R2), and FXa-specific chromogenic substrate powder (R3) contained in the kit were each completely dissolved by adding 1 mL of sterile water. These reagents are normally kept refrigerated and were shaken in a constant temperature shaking incubator (Model. No. M-RB-022UP, Taitec) at 100 rpm and 25° C. for 30 minutes before the assay. The required volumes of the reagents were dispensed, and diluted 5-fold with Tris NaCl EDTA PEG buffer-pH 8.40 in the case of the R1 and R2 solutions, or diluted 5-fold with sterile water in the case of the R3 solution, respectively. Immediately prior to use in the assay, the reagent solutions were incubated at 37° C.

The culture supernatant was centrifuged at 13,200 g for 5 minutes before the assay, and 10 μL of the supernatant was used as a sample. The standard solutions (10 μL) and the sample (10 μL) were transferred to a 384-well ELISA plate (Cat. No. 3711-384, Iwaki) and incubated at 37° C. for 5 minutes. The 384-well ELISA plate was used here because it makes it easier to mix the reagents added to the plate compared with a larger container. The R1 solution (10 μL) was added to each well, and the plate was incubated at 37° C. for 2 minutes. After the reaction, the R2 solution (10 μL) was added and the plate was incubated at 37° C. for exactly 2 minutes. After the reaction, the R3 solution (10 μL) was added and the plate was incubated at 37° C. for exactly 2 minutes to cause the color development reaction. After the reaction, 20 μL of 20% aqueous acetic acid (Cat. No. 017-00256, Wako) was added to terminate the reaction, and absorbance (405 nm) was measured with a plate reader (Model. No, Enspire, PerkinElmer).

### (b) Testzym Heparin S Assay Kit

When using the Testzym Heparin S assay kit, the heparin standard solution was prepared by using heparin sodium (Cat. No. 081-00136, Wako, Lot: WDR1808). The antithrombin III powder, FXa powder, and FXa-specific chromogenic substrate powder contained in the kit were completely dissolved in sterile water of the volumes specified by the kit. Only the required volumes of the solutions were dispensed and incubated at 37° C. immediately before use in the assay. As for the standard, a 1000 IU/mL solution was first prepared with 0.85% NaCl solution, and then diluted 10-fold twice to prepare a 10 IU/ml solution. The 10 IU/ml solution was further diluted 50-fold with the buffer packaged in the kit to prepare a 0.2 IU/ml solution. This was mixed with the buffer and human plasma packaged in the kit on a 96-well ELISA plate (Cat. No. 3801-096, Iwaki) and used as a standard.

The culture supernatant was centrifuged at 13,200 g for 5 minute before the assay, and 5 μL of the supernatant was used as a sample. The sample (5 μl) was transferred to a 96-well ELISA plate and 40 μl of the buffer packaged in the kit was added to make a total volume of 45 μl. The standard and sample were then incubated at 37° C. for 5 minutes. To the standard and sample, 5 μl of the antithrombin III solution was added, and the mixture was incubated at 37° C. for 6 minutes. After the reaction, 25 μL of the FXa solution was added and the mixture was incubated at 37° C. for exactly 30 seconds. After the reaction, 50 μL of the FXa-specific chromogenic substrate solution was added and the mixture was incubated at 37° C. for exactly 3 minutes to cause the color development reaction. After the reaction, 75 μL of 20% aqueous acetic acid was added to terminate the reaction. Then, 100 μl of each sample was transferred to a 384-well ELISA plate and absorbance (405 nm) was measured with a plate reader (Model No. Enspire, PerkinElmer).

TABLE 1
Preparation of standards

| | | | | | |
|---|---|---|---|---|---|
| Activity of standard [IU/ml] | 0 | 0.2 | 0.4 | 0.6 | 0.8 |
| 0.2 IU/ml solution [μl] | 0 | 5 | 10 | 15 | 20 |
| Buffer [μl] | 40 | 35 | 30 | 25 | 20 |
| Human plasma [μl] | 5 | 5 | 5 | 5 | 5 |
| Total [μl] | 45 | 45 | 45 | 45 | 45 |

(2) Method for Measuring sGAG Amount in Culture Supernatant Using sGAG Assay Kit

The inoculation and collection methods of the culture supernatant samples used were the same as those used for the anti-FXa activity assay. Established cell clones or cells of each cell line were inoculated into FreeStyleCHO-S Expression Medium at a cell density of 0.6×10⁶ cells/well in a volume of 2 mL/well using a 6-well plate on day 0. Culture was performed without medium change and subculture, and the culture supernatant was collected on day 3. The undiluted culture supernatant was used as a sample. The concentrations of sulfated glycosaminoglycans (sGAG) contained in the obtained samples were quantified by using Blyscan Glycosaminoglycan Assay Kit (Cat. No. B1000, QBS). The assay was performed according to the manual of the kit. The procedure is shown below.

The culture supernatant was centrifuged at 13,200 g for 5 minutes before the assay and 100 μl was used as the sample. The sample or standard (100 μL) was placed in a 1.5-mL microtube, 1,000 μL of Blyscan Dye (1,9-dimethyl-methylene blue) Reagent was added, and then they were mixed at 100 rpm for 30 minutes at room temperature (Model No. M-BR-022 UP, Maximizer, Taitec). The mixture was centrifuged (20,000×g, 10 minutes, 4° C.) to obtain a pellet of the reaction product of sGAG and Blyscan Dye Reagent, then the supernatant was removed, and the pellet was dissolved with 500 μL of Dye Dissociation Reagent. Since the pellet is easily detached from the wall of the microtube at the time of removing the supernatant, a certain volume of the supernatant was first removed by decantation, and then the remaining supernatant was carefully removed with a micropipette so that the supernatant did not remain as much as possible. Finally, the solution was centrifuged (20,000×g, 10 minutes, 4° C.), 200 μL of the solution was transferred to a 6-well ELISA plate (Cat. No. 3801-096, Iwaki), and absorbance (656 nm) was measured with a plate reader (Model No. Enspire, PerkinElmer).

### (3) Test for Significant Differences in Assay Results

Concerning the results obtained in the assays, for those obtained from experiments performed with n=3 or more, significant differences based on the results of controls obtained in the control experiments (e.g., non-treatment condition) were calculated as appropriate. First, the F-test was performed on the two data groups of control results and obtained results. The significance level was set at 0.1, and when the obtained two-tailed probability was greater than 0.1, it was determined that the results were considered to have equal variances, and when less than 0.1, unequal variances. Then, the Student's t-test (t-test) was performed on the two data groups of the results of the control and obtained results. On the basis of the determination for equal/unequal variances, a two-tailed test was performed, and when the significance level was greater than 0.05, it was determined that there was no significant difference, or when 0.05 or smaller, it was determined that there was significant difference.

### 3. Acquisition of Expression Vectors for Transient Gene Expression

The expression vectors used in the transient expression experiments were purchased from ORIGEN and Horizon Discovery. For the vectors obtained in the form of E. coli frozen stock, culture and plasmid DNA extraction were performed according to the protocol described below. As for those obtained as plasmid DNA, they were transformed into competent cells, then the cells were similarly cultured, and the plasmid DNA was extracted. The target gene sequences were confirmed by sequencing analysis (Prism 3130 Genetic Analyzer, Applied Biosystems) before use in the experiments.

### 4. Method for Producing Vector

(1) Preparation of E. coli Competent Cells for Transformation

Cells of an E. coli strain (DH5a, Takara) were inoculated on LB agar medium (1% polypeptone, 1% NaCl, 0.5% yeast extract, and 2% agar) and cultured overnight at 37° C. Single colonies were then isolated from the agar medium and transferred to 30 mL of LB medium for competent cell preparation (1% polypeptone, 1% NaCl, 0.5% yeast extract, 0.02 M MgSO₄·7H₂O, 0.02 M MgCl₂·6H₂O (pH 7.2 to 7.3)) and shaking-cultured. The culture was terminated approximately 3 hours after the turbidity of the culture medium (OD600) reached 0.45. The bacterial cells were then collected by centrifugation (3,000×g, 10 minutes, 4° C.) and the supernatant was discarded. To the obtained bacterial cells, 10 mL of a transformation buffer (10 mM PIPES, 15 mM CaCl₂·2H₂O, 250 mM KCl, 55 mM MnCl₂·4H₂O (pH 6.7 to 6.8)) was added to gently suspend the cells, and the suspension was allowed to stand on ice for 10 minutes. Further, the bacterial cells were collected by centrifugation at 3,000×g for 10 minutes at 4° C., the supernatant was discarded, the resulting pellet was suspended in 2.4 mL of the transformation buffer and 100 μL of DMSO, and the suspension was dispensed in 100 μL portions into microcentrifuge tubes, frozen in liquid nitrogen and stored at −80° C.

### (2) Transformation Using Competent Cells

A sample solution (10 μL) containing plasmid DNA (several ng) and 100 μL of the E. coli competent cell suspension were added to a microcentrifuge tube, gently mixed by pipetting, and allowed to stand on ice for 30 minutes. Heat shock was applied for 45 seconds in a 42° C. thermostatic bath, and then the cell suspension was left on ice for 5 minutes. Then, 0.4 mL of LB medium was added to the mixture of the plasmid and competent cells to make the total volume of 0.5 mL, and culture was performed at 37° C. for 1 hour. The entire culture medium was then inoculated on LB agar medium containing antibiotic by evenly spreading it with a bacteria spreader. The bacterial cells were cultured overnight at 37° C. to obtain colonies of cells with the target plasmids. When a plasmid that allows blue-white selection was used, the E. coli cells were inoculated after application of X-gal (20 μg/mL, 50 μL) and IPTG (100 mM, 25 μL), and white colonies that were considered to have the objective plasmid were obtained.

(3) Preparation of Glycerol Stock of E. coli

The transformed E. coli cells carrying the target DNA (genetically modified E. coli, 200 μL) were suspended in 800 μL of a 50% glycerol solution and stored at −80° C. The 50% glycerol solution was prepared by mixing glycerol (Cat. No. 075-00616, Wako) and sterile water at a volume ratio of 1:1.

### (4) Small-Scale Preparation of Plasmid DNA (Alkali Method)]

Transformed E. coli colonies were isolated, and each was added to 2 mL of the LB liquid medium and shaking-cultured overnight at 37° C. for sufficient growth. On the next day, 1 mL of the E. coli culture was taken in a microcentrifuge tube, and centrifuged (6,000×g, 1 minute, room temperature), and the supernatant was removed to collect the bacterial cells. To the bacterial cells, 100 μL of a glucose solution (50 mM glucose, 25 mM Tris-HCl (pH 8.0), and 10 mM EDTA) was added, and the cells were suspended by using a vortex mixer. Then, an alkali-SDS solution (0.2 N NaOH, 1% (w/v) SDS) was added, and the microcentrifuge tube was gently inverted several times to lyse the bacterial cells. Further, 150 μL of 3 M potassium acetate (pH 4.5) was added, and the microcentrifuge tube was gently inverted several times to neutralize the cell lysate. Then, 450 μL of a phenol-chloroform mixture was added and mixed well to emulsify the cell lysate, and the emulsion was centrifuged (12,000×g, 5 minutes, room temperature) to separate it into an organic layer and an aqueous layer containing DNA. About three-quarters of the aqueous layer was transferred to another microcentrifuge tube, 300 μL of isopropanol was added, and the mixture was vortexed, and centrifuged (20,000×g, 10 minutes, 4° C.) to precipitate DNA. Then, the supernatant was removed, and the DNA pellet was washed by adding 200 μL of 70% (v/v) ethanol, and centrifuging the mixture (20,000×g, 5 minutes, 4° C.). The supernatant was discarded and the pellet was dried on a heat block (5 minutes, 55° C.). Finally, the DNA pellet was well suspended and dissolved in 30 μL of ultrapure water containing 1% (v/v) RNase by pipetting or other means.

(5) Digestion of DNA with Restriction Enzymes

The plasmid solution, a restriction enzyme, and a buffer corresponding to the restriction enzyme were mixed in required volumes or amounts in a microcentrifuge tube, and the reaction was allowed at 37° C. for 2 hours to digest the DNA. When DNA fragments were purified in this process, a plasmid solution containing 5 μg of the plasmid, a restriction enzyme and a buffer were used to obtain a total volume of the reaction solution of 40 μL. When restriction enzyme analysis was performed, a plasmid solution containing 300 ng of plasmid was used in the same way to obtain the total volume of the reaction solution of 10 μL.

### (6) Confirmation of Restriction Enzyme Fragments by Agarose Gel Electrophoresis

A loading buffer was added to the DNA solution sample, and the sample was electrophoresed in agarose gel of a concentration appropriate for the molecular weights of the DNA fragments. Markers of which electrophoresis patterns were already known (lamda-HindIII-digested or phiX174-HincII-digested markers) were simultaneously electrophoresed, and the position of the band of the sample was confirmed.

(7) Purification of DNA Fragments from Agarose Gel

After the agarose gel electrophoresis, DNA fragments were stained with ethidium bromide, and the portion of the desired band was cut out with a cutter while confirming the DNA fragments under UV irradiation. The cut agarose gel was then cut into small pieces and transferred to a microcentrifuge tube. The DNA fragments were then purified by using DNA Fragment Purification Kit (Cat. No. NPK-601, MagExtractor-PCR & Gel Clean Up-, Toyobo). The operation was performed according to the protocol attached to the kit.

(8) DNA Ligation with DNA Ligase (Ligation)

A DNA solution containing the plasmid vector and insert DNA (5 μL) was prepared, and ligation of DNA was performed by using LigaFast™ Rapid DNA Ligation System (Cat. No. M8221, Promega) at 16° C. for 12 hours or at room temperature for 2 hours. The operation was performed according to the protocol attached to the kit.

### (9) Mass Extraction of Plasmid DNA

The transformed E. coli cells carrying the target DNA were added to 2 mL of LB liquid medium and cultured at 37° C. for 8 hours with shaking. Then, 100 μL of this culture was added to 40 mL of LB liquid medium and culture was further continued for 1 to 16 hours with shaking. The culture medium was then centrifuged (6,000×g, 15 minutes, 4° C.), and the supernatant was removed to collect the bacterial cell pellet. Then, a plasmid extraction kit (QIAfilter Plasmid Midi Kits (Cat. No. 12445, Qiagen)) was used for mass extraction of plasmids. The operation was performed according to the protocol attached to the kit, which will be briefly explained below.

The bacterial cell pellet was resuspended in 4 mL of Buffer P1. Then, 4 mL of Buffer P2 was added, and the suspension was sufficiently mixed by 4 to 6 times of vigorous inversion, and left at room temperature for 10 minutes. The lysate was poured into a QIAfilter Cartridge and incubated at room temperature for 10 minutes. Buffer QBT (4 mL) was added to QIAGEN-tip 100, and the column was left to stand to allow flow out of the buffer until empty for equilibration. A plunger was placed in QIAfilter Midi Cartridge, and the cell lysate was poured into the pre-equilibrated QIAGEN-tip 100 and filtered. The lysate was allowed to permeate the resin by free gravity fall. The QIAGEN-tip was washed twice with 10 mL of Buffer QC. DNA was eluted with 5 mL of Buffer QF, and 3.5 mL of isopropanol (room temperature) was added to the eluate to precipitate DNA. After mixing, the mixture was immediately centrifuged at 15,000×g and 4° C. for 30 minutes. The supernatant was carefully decanted and the DNA pellet was incubated at room temperature for 5 minutes. The DNA pellet was washed with 2 mL of 70% ethanol and centrifuged at 4° C. and 15,000×g for 10 minutes. The supernatant was similarly carefully decanted, and the pellet was dried for 5 to 10 minutes, and then re-dissolved in about 50 μL of TE buffer to prepare a plasmid solution.

### 5. Preparation of Hs6st3 Gene Expression Vector

### (1) Determination of Target Gene

The inventors of the present invention focused on heparan sulfate-glucosamine 6-sulfotransferase 3 (Hs6st3, Accession No. NM_015820) derived from mouse, which showed the highest enhancement of the anti-FXa activity in heparin-like substance-producing cell culture supernatant under transient expression conditions, and used it as a target gene.

### (2) Method for Producing Hs6st3 Gene Expression Vector

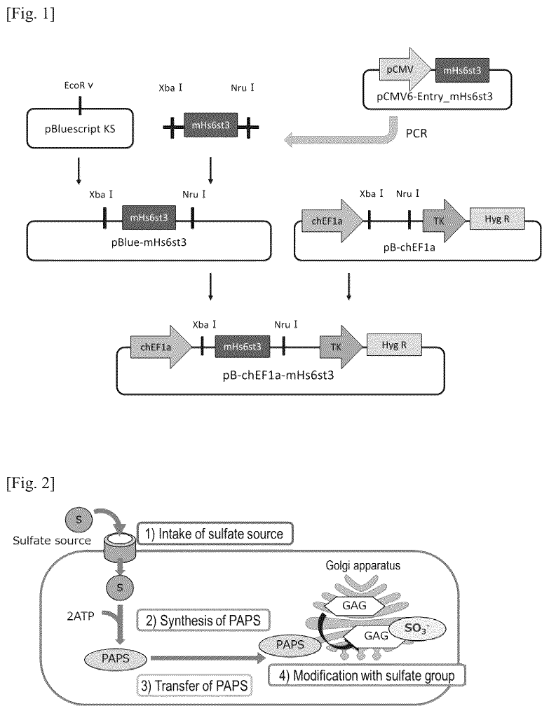

The procedure for preparing the Hs6st3-expressing transposon vector is shown in FIG. 1. The following is a brief description of the procedure for preparing the vector. The Hs6st3 gene was amplified by PCR using the Hs6st3 expression plasmid vector (ORIGEN, Catalog ID: MR217272) used in the experiments under transient expression conditions as a template. The operations of PCR were performed by using KOD-Plus-Neo (Cat. No. KOD-401, Toyobo) according to the manual of the kit. The primers and PCR conditions used are shown in the following table. The PCR products were electrophoresed on agarose gel, and after confirming amplification of DNA in the bands, collected by using a DNA purification kit (Mag Extractor-PCR & Gel clean up-). The PCR products were ligated with pBluescript II KS (−) treated with EcoRV, E. coli competent cells were transformed with the ligation products, and E. coli cells having the target gene sequence were cloned. After mass extraction of plasmid DNA, only the target gene region was cut out with XbaI and NruI. The target gene region was ligated with the transposon vector PB/chEFlaEBNA1-TKHyg, which was similarly treated with the restriction enzymes, to obtain PB-chEFla-Hs6st3. The target gene sequence was then confirmed by sequencing analysis (Prism 3130 Genetic Analyzer, Applied Biosystems). The sequencing analysis was entrusted to Hokkaido System Science.

TABLE 2
Primer sequences

| Primer (5′→3′) | Sequence | |
|---|---|---|
| mHs6st3_XbaI_FW | AAAATCTAGAGCCGCCACCATGGATGAAAGGTTCAACAAGT | SEQ ID NO: 35 |
| | GGCTGC | |
| mHs6st3_DraI_NruI_RV | TTTAAATCGCGATTATCACCATCTGACCACTTGGCTGTTGTA | SEQ ID NO: 36 |
| | GTCC | |

TABLE 3
PCR conditions

| | Initial | Thermal | | | |
|---|---|---|---|---|---|
| | dena- | dena- | | | Final |
| | turation | turation | Annealing | Extension | hold |
| Cycle (No.) | 1 | | 30 | | 1 |
| Temperature | 98 | 98 | 64 | 68 | 4 |
| (° C.) | | | | | |
| Time (seconds) | 120 | 10 | 30 | 45 | ∞ |

### 6. Methods for Gene Transfer and Establishment of Recombinant Cells

### (1) Transfection (Lipofection)

The day before transfection, transgenic CHO-S/NH-SDC cells cultured in a serum-free medium were inoculated in F12+10% FBS on a 6-well plate (Cat. No. 130184, Thermo) at a density of 1.2×10⁶ cells/well in a volume of 2 mL/well. On the next day, a DNA solution was prepared by adding 250 μL of Opti-MEM (Cat. No. 31985-070, Invitrogen) to 4,000 ng of the vector. Further, 12.5 μL of Lipofectamine 2000 (LF2000, Cat. No. 11668-019, Invitrogen) was added to 250 μL of Opti-MEM (Invitrogen) and allowed to stand for 5 minutes. These two solutions were mixed and allowed to stand at room temperature for 30 minutes. During these operations, the medium of the cells cultured on the 6-well plate was changed to 2 ml of serum-free F12 medium. Then, the DNA/LF2000 mixture was added in a volume of 500 μL/well and the cells were cultured at 37° C. After 6 hours, the medium was changed to the serum-free medium. For experiments by transient expression, the culture was left for 48 hours after the transfection, and the culture supernatant was collected. To obtain stably expressing strains, the cells were left for 48 hours after the transfection, inoculated again in F12+10% FBS on a normal TC dish or plate, cultured and selected under an adhered condition, as already described.

(2) Screening with Drug

To obtain stably expressing strains, transfected cells were subcultured on a 6-well plate 48 hours after the transfection, and drug selection was performed by using a hygromycin B solution (Cat. No. 080-07683, Wako, final concentration 400 μg/mL). While the cells were subcultured 4 to 5 times, cell selection was performed, and drug-resistant cells were obtained after approximately 14 days.

### (3) Cloning of Cells by Limiting Dilution

The cell suspension was diluted stepwise to a density corresponding to 1 cell/well, and inoculated onto a collagen-coated 96-well plate (Cat. No. 4860-010, Iwaki). The medium used was F12+10% FBS medium. The cells were cultured with confirmation of presence and proliferation of the cells as appropriate, and single clones were obtained from the bulk cells.

### Results

### 1. Anti-FXa Activity Observed by Transient Expression

The genes involved in the steps of the sulfation pathway were introduced into heparin-like glycan-secreting cell clones to attempt enhancement of sulfation and anticoagulant activity of heparin-like substances. Specifically, the CHO-S/NH-SDC cells established in the laboratory of the inventors of the present invention (see Patent document 5, 1.2× 10⁶ cells/well, 2 mL/well) were transfected with a vector for transient expression containing a candidate gene, and left in a serum-free medium for 48 hours, then the culture supernatant was collected, and the anticoagulant factor Xa activity (anti-FXa activity) of the culture supernatant was evaluated. The sulfation pathway is shown in FIG. 2, and the genes involved in the respective steps are listed below.

TABLE 4

| Step | Name of gene | Function |
|---|---|---|
| 1) | SLC26A1 | Transfer of sulfate |
| | SLC26A2 | ions into cells |
| | SLC13A1 | |
| | SLC13A4 | |
| 2) | PAPSS1 | Synthesis of PAPS from |
| | PAPSS2 | sulfate ions |
| 3) | SLC35B2 | Transfer of PAPS to |
| | SLC35B3 | Golgi apparatus |
| 4) | Glce | Conversion of sugar chain |
| | NDST1 | N-Deacetylation/N-sulfation |
| | Hs2st | 2-O-Sulfation |
| | Hs6st1 | 6-O-Sulfation |
| | Hs6st2 | |
| | Hs6st3 | |
| | Hs3st5 | 3-O-Sulfation |

Details of the candidate genes are shown in the following table.

TABLE 5

| Symbol | Name of | | | SEQ |
|---|---|---|---|---|
| of gene | gene | Alias | Details | ID NO |
| SLC26A1 | Solute carrier family | Sulfate anion | Mouse Slc26a1 | 1 |
| | 26 member 1 | transporter 1 | (BC032151.1) | |
| SLC26A2 | Solute carrier family | Sulfate transporter | Mouse Slc26a2 | 3 |
| | 26 member 2 | | (BC028345.1) | |
| SLC13A1 | Solute carrier family | Na(+)/Sulfate | Mouse Slc13a1 cDNA | 5 |
| | 13 member 1 | cotransporter | (BC040789.1) | |
| SLC13A4 | Solute carrier family | Na(+)/Sulfate | Mouse Slc13a4 cDNA | 7 |
| | 13 member 4 | cotransporter SUT-1 | (BC089161.1) | |
| PAPSS1 | 3′-Phosphoadenosine | Bifunctional | Human PAPSS1 cDNA | 9 |
| | 5′-phosphosulfate | 3′-phosphoadenosine | (BC040940.1) | |
| | synthase 1 | 5′-phosphosulfate | | |
| | | synthase 1 | | |
| PAPSS2 | 3′-Phosphoadenosine | Bifunctional | Mouse Papss2 cDNA | 11 |
| | 5′-phosphosulfate | 3′-phosphoadenosine | (NM_011864.2) | |
| | synthase 2 | 5′-phosphosulfate | | |
| | | synthase 2 | | |
| SLC35B2 | Solute carrier family | Adenosine 3′-phospho- | Mouse Slc35b2 cDNA | 13 |
| | 35 member B2 | 5′-phosphosulfate | (BC036992.1) | |
| | | transporter 1 | | |
| SLC35B3 | Solute carrier family | Adenosine 3′-phospho | Mouse Slc35b3 cDNA | 15 |
| | 35 member B3 | 5′-phosphosulfate | (BC006881.1) | |
| | | transporter 2 | | |
| Glce | Glucuronic acid | D-Glucuronyl | Mouse Glce cDNA | 17 |
| | epimerase | C5-epimerase | (NM_033320.5) | |
| NDST1 | N-Deacetylase and | Glucosaminyl | Human NDST1 cDNA | 19 |
| | N-sulfotransferase 1 | N-deacetylase/N- | (BC012888.1) | |
| | | sulfotransferase 1 | | |
| NDST2 | N-Deacetylase and | Glucosaminyl | Human NDST2 cDNA | 21 |
| | N-sulfotransferase 2 | N-deacetylase/N- | (NM_003635) | |
| | | sulfotransferase 2 | | |
| Hs2st | Heparan sulfate 2-O- | 2-O-Sulfotransferase | Human HS2ST1 cDNA | 23 |
| | sulfotransferase | | (BC108735.1) | |
| Hs6st1 | Heparan sulfate 6-O- | | Mouse Hs6st1 cDNA | 25 |
| | sulfotransferase 1 | | (BC052316.1) | |
| Hs6st2 | Heparan sulfate 6-O- | | Mouse Hs6st2 cDNA | 27 |
| | sulfotransferase 2 | | (BC037659.1) | |
| Hs6st3 | Heparan sulfate 6-O- | | Mouse Hs6st3 cDNA | 29 |
| | sulfotransferase 3 | | (NM_015820.2) | |
| Hs3st1 | Heparan sulfate 3-O- | Heparan sulfate | Mouse Hs3st1 cDNA | 31 |
| | sulfotransferase 1 | (glucosamine) | (NM_010474) | |
| | | 3-O-sulfotransferase 1 | | |
| Hs3st5 | Heparan sulfate- | Heparan sulfate | Mouse Hs3st5 cDNA | 33 |
| | glucosamine 3- | (glucosamine) | (NM_001081208.2) | |
| | sulfotransferase 5 | 3-O-sulfotransferase 5 | | |

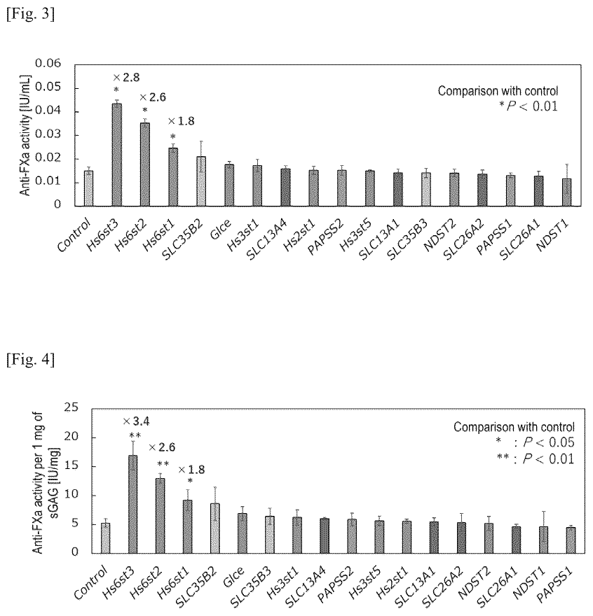

The results are shown in FIG. 3. Among the candidate genes, the anti-FXa activity in the culture supernatant was significantly enhanced when the genes encoding Hs6st3, Hs6st2, and Hs6st1, three enzymes that contribute to 6-O-sulfation in the modification of sulfate group in the sulfation pathway, were introduced.

### 2. Calculation of Specific Activity

The sulfated glucosaminoglycan (sGAG) concentration in the culture supernatant was not significantly enhanced by transient expression of any of the genes. Therefore, specific activity (anti-FXa activity per mg of sGAG) in the culture supernatant was determined for each case. The results are shown in FIG. 4. It was found that transfection of the Hs6st3 gene is important for the production of heparin-like substances. Expression of the Hs6st3 gene in the CHO cells has not been confirmed, like the genes encoding glucosaminyl N-deacetylase/N-sulfotransferase (NDST2), and heparan sulfate 3-O-sulfotransferase 1 (Hs3st1), which have been transfected previously (Patent document 5).

### 3. Specific Activity of Hs6st3-Expressing Cells

The Hs6st3 gene, which was highly effective in the previous experiment, was introduced into the CHO-S/NH-SDC cells by lipofection, followed by drug selection with hygromycin to obtain a stably expressing strain. The obtained cells were inoculated on a 6-well plate at a density of 0.6×10⁶ cells/well (2 mL/well) and cultured in FreeStyleCHO-S Expression Medium for 3 days without medium change and subculture, and then the culture supernatant was collected. The Anti-FXa activity of the collected culture supernatant was determined.

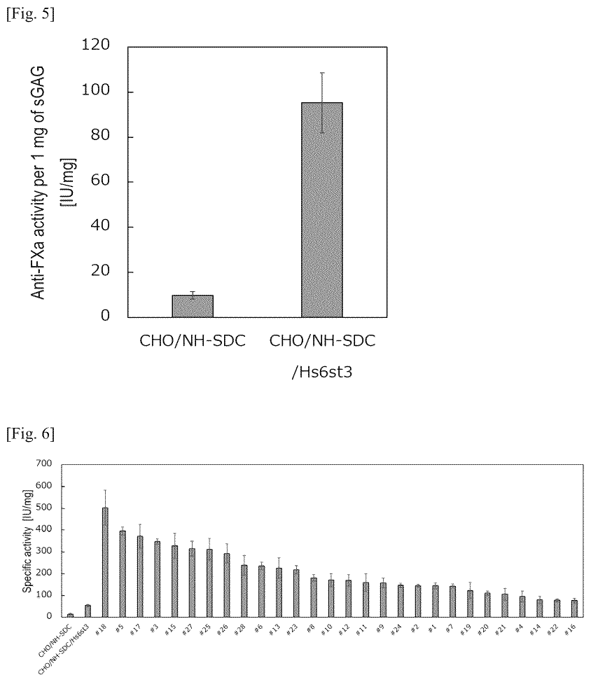

The results are shown in FIG. 5. The anti-FXa activity per 1 mg of sGAG (specific activity) in the cells transfected with the Hs6st3 gene (CHO/NH-SDC/Hs6st3) after the culture was 95 IU/mg, a 9.7-fold increase in specific activity compared with the original CHO-S/NH-SDC cells.

The specific activity of commercial heparin is 248 IU/mg. The heparin-like substance produced by CHO/NH-SDC/Hs6st3 has about 38% of that activity.

### 4. Evaluation of Hs6st3-Expressing Cell Clones

Cells transfected with the Hs6st3 gene obtained above were diluted stepwise and inoculated on a collagen-coated plate at a density of 1 cell/well. For the culture, FBS medium containing 10% serum was used. The culture was performed with confirmation of presence and proliferation of the cells as appropriate, and 28 single clones were obtained from the bulk cells.

The specific activities of the obtained clones are shown in FIG. 6. A 1.5-to 9-fold increase in specific activity from that of the bulk cells could be confirmed. It can be said that an anticoagulant with the activity comparable to that of commercial heparin could be obtained from the recombinant CHO cells.

### [Evaluation of CHO/NH-SDC/Hs6st3 (#17)]

The anti-FXa activity of the heparin-like substance contained in the culture supernatant of the stably expressing strain obtained in the previous experiment was measured as described above, and the anti-FIIa activity of the same was evaluated by the method described below.

### (1) Method

### (a) BIOPHEN HEPARIN ANTI-IIa (2 Stages)

When BIOPHEN HEPARIN ANTI-IIa (2 stages) is utilized, BIOPHEN UFC Calibrator (Cat. No. 222301-RUO), which contains samples of known activity values, was used for the preparation of a calibration curve. The five kinds of powder samples contained in the BIOPHEN UFC Calibrator kit were each dissolved by adding 1 mL of sterile water. These solutions were diluted 15-fold by adding Tris EDTA NaCl BSA buffer-pH 8.40 (Cat. No. AR031K) to prepare five kinds of standard solutions. Antithrombin III powder (R1), FIIa powder (R2), and FIIa-specific chromogenic substrate powder (R3) contained in the kit were each completely dissolved by adding 1 mL of sterile water. These reagents are normally kept refrigerated and were shaken in a constant temperature shaking incubator (Model. No. M-RB-022UP, Taitec) at 100 rpm and 25° C. for 30 minutes before the assay. The required volumes of the reagents were dispensed, and diluted 5-fold with Tris NaCl EDTA PEG buffer-pH 8.40 in the case of the R1 and R2 solutions, or diluted 5-fold with sterile water in the case of the R3 solution, respectively. Immediately prior to use in the assay, the reagent solutions were incubated at 37° C.

The culture supernatant was centrifuged at 13,200 g for 5 minutes before the assay, and 10 μL of the supernatant was used as a sample. The standard solutions (10 μL) and the sample (10 μL) were transferred to a 96-well assay plate (Cat. No. 3882-096, Iwaki) and incubated at 37° C. for 5 minutes. The 96-well assay plate was used here because it makes it easier to mix the reagents added to the plate compared with a larger container. The R1 solution (10 μL) was added to each well, and the plate was incubated at 37° C. for 2 minutes. After the reaction, the R2 solution (10 μL) was added and the plate was incubated at 37° C. for exactly 2 minutes. After the reaction, the R3 solution (10 μL) was added and the plate was incubated at 37° C. for exactly 2 minutes to cause the color development reaction. After the reaction, 20 μL of 20% aqueous acetic acid (Cat. No. 017-00256, Wako) was added to terminate the reaction, 50 μL each of the reaction mixtures were transferred to a 384-well ELISA plate (Cat. No. 3711-384, Iwaki), and absorbance (405 nm) was measured with a plate reader (Model. No. Enspire, PerkinElmer).

### (2) Results

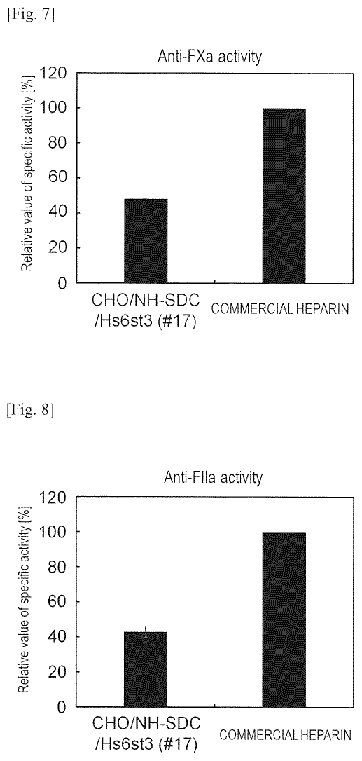

The results are shown in FIGS. 7 and 8. The specific activity of the heparin-like substance contained in the culture supernatant of the #17 clone of CHO/NH-SDC/Hs6st3 was expressed as a relative value based on that of the commercial heparin (specific activity 170 IU/mg). The heparin-like substance produced by CHO/NH-SDC/Hs6st3 (#17) had an activity more than 40% of that of the commercial heparin, and showed anti-FXa/anti-FIla values comparable to those of the commercial heparin. Low molecular weight heparins and synthetic heparins showed only the anti-FXa activity and no anti-FIIa activity. The fact that both activities were detected in such degrees comparable to those of the commercial heparin indicates that a substance having a structure similar to that of the commercial heparin may be produced.

### Sequences Listed in Sequence Listing

SEQ ID NO: 1 Mouse Slc26a1 (BC032151.1)
SEQ ID NO: 2 Mouse Slc26a1, PRT sequence
SEQ ID NO: 3 Mouse Slc26a2 (BC028345.1)
SEQ ID NO: 4 Mouse Slc26a2, PRT sequence
SEQ ID NO: 5 Mouse Slc13a1 cDNA (BC040789.1)
SEQ ID NO: 6 Mouse Slc13a1, PRT sequence
SEQ ID NO: 7 Mouse Slc13a4 cDNA (BC089161.1)
SEQ ID NO: 8 Mouse Slc13a4, PRT sequence
SEQ ID NO: 9 Human PAPSS1 cDNA (BC040940.1)
SEQ ID NO: 10 Human PAPSS1, PRT sequence
SEQ ID NO: 11 Mouse Papss2 cDNA (NM_011864.2)
SEQ ID NO: 12 Mouse Papss2, PRT sequence
SEQ ID NO: 13 Mouse Slc35b2 cDNA (BC036992.1)
SEQ ID NO: 14 Mouse Slc35b2, PRT sequence
SEQ ID NO: 15 Mouse Slc35b3 cDNA (BC006881.1)
SEQ ID NO: 16 Mouse Slc35b3, PRT sequence
SEQ ID NO: 17 Mouse Glce cDNA (NM_033320.5)
SEQ ID NO: 18 Mouse Glce, PRT sequence
SEQ ID NO: 19 Human NDST1 cDNA (BC012888.1)
SEQ ID NO: 20 Human NDST1, PRT sequence
SEQ ID NO: 21 Human NDST2 cDNA (NM_003635)
SEQ ID NO: 22 Human NDST2, PRT sequence
SEQ ID NO: 23 Human HS2ST1 cDNA (BC108735.1)
SEQ ID NO: 24 Human HS2ST1, PRT sequence
SEQ ID NO: 25 Mouse Hs6st1 cDNA (BC052316.1)
SEQ ID NO: 26 Mouse Hs6st1, PRT sequence
SEQ ID NO: 27 Mouse Hs6st2 cDNA (BC037659.1)
SEQ ID NO: 28 Mouse Hs6st2, PRT sequence
SEQ ID NO: 29 Mouse Hs6st3 cDNA (NM_015820.2)
SEQ ID NO: 30 Mouse Hs6st3, PRT sequence
SEQ ID NO: 31 Mouse Hs3st1 cDNA (NM_010474)
SEQ ID NO: 32 Mouse Hs3st1, PRT sequence
SEQ ID NO: 33 Mouse Hs3st5 cDNA (NM_001081208.2)
SEQ ID NO: 34 Mouse Hs3st5, PRT sequence
SEQ ID NO: 35 mHs6st3_XbaI_FW
SEQ ID NO: 36 mHs6st3_DraI NruI RV
SEQ ID NO: 37 Human extracellular domain of SDC, nucleotide sequence (corresponding to SEQ ID NO: 6 in Patent document 5)
SEQ ID NO: 38 Human extracellular domain of SDC, PRT sequence (corresponding to SEQ ID NO: 1 in Patent document 5)
SEQ ID NO: 39 Human NDST2, nucleotide sequence (corresponding to SEQ ID NO: 4 in Patent document 5)
SEQ ID NO: 40 Human NDST2, PRT sequence (corresponding to SEQ ID NO: 2 in Patent document 5)
SEQ ID NO: 41 Mouse Hs3st1, nucleotide sequence (corresponding to SEQ ID NO: 4 in Patent document 5)
SEQ ID NO: 42 Hs3st1, PRT sequence (corresponding to SEQ ID NO: 3 in Patent document 5)
SEQ ID NO: 43 Human heparan sulfate 6-O-sulfotransferase 3 (HS6ST3) (NM_153456)
SEQ ID NO: 44 Human HS6ST3, PRT sequence
SEQ ID NO: 45 Human heparan sulfate 3-O-sulfotransferase 1 (HS3ST1) (NM_005114)
SEQ ID NO: 46 Human HS3ST1, PRT sequence

1. A method for producing a heparin-like substance, the method comprising culturing an animal cell to which the followings are introduced to obtain a culture supernatant containing a heparin-like substance:
a polynucleotide encoding glucosaminyl N-deacetylase/N-sulfotransferase (NDST2);
a polynucleotide encoding heparan sulfate 3-O-sulfotransferase 1 (Hs3st1); —a polynucleotide encoding the extracellular domain of syndecan (SDC); and
a polynucleotide encoding 6-O-sulfotransferase.

2. The method according to claim 1, wherein the 6-O-sulfotransferase is any selected from the group consisting of heparan sulfate 6-O-sulfotransferase 1 (Hs6st1), heparan sulfate 6-O-sulfotransferase 2 (Hs6st2), and heparan sulfate 6-O-sulfotransferase 3 (Hs6st3).

3. The method according to claim 1, wherein the 6-O-sulfotransferase is Hs6st3.

4. The method according to claim 1, wherein the animal cell is CHO cell.

5. A recombinant heparin-like substance produced by the method according to claim 1, which has an anticoagulant factor Xa activity per mg of 200 IU/mg or higher.

6. A recombinant CHO cell introduced with at least the followings:
a polynucleotide encoding NDST2;
a polynucleotide encoding Hs3st1;
a polynucleotide encoding the extracellular domain of SDC; and
a polynucleotide encoding 6-O-sulfotransferase.

7. The cell according to claim 6, wherein the 6-O-sulfotransferase is any selected from the group consisting of Hs6st1, Hs6st2, and Hs6st3.

8. The cell according to claim 6, wherein the 6-O-sulfotransferase is Hs6st3.

9. The cell according to claim 6, wherein the polynucleotide encoding 6-O-sulfotransferase is any of the followings:
(A) a polynucleotide consisting of the sequence of SEQ ID NO: 29 or 43;
(B) a polynucleotide that hybridizes with a polynucleotide consisting of a sequence complementary to the sequence of SEQ ID NO: 29 or 43 under stringent conditions and encodes a protein having a function to enhance the anticoagulant factor Xa activity of a heparin-like substance produced by a heparin-like substance-producing animal cell;
(C) a polynucleotide consisting of a sequence having an identity of 90% or higher to the sequence of SEQ ID NO: 29 or 43 and encoding a protein having a function to enhance the anticoagulant factor Xa activity of a heparin-like substance produced by a heparin-like substance-producing animal cell;
(D) a polynucleotide encoding a protein consisting of the amino acid sequence of SEQ ID NO: 30 or 44;
(E) a polynucleotide encoding a protein consisting of an amino acid sequence derived from the amino acid sequence of SEQ ID NO: 30 or 44 by substitution, deletion, insertion, and/or addition of multiple amino acids and having a function to enhance the anticoagulant factor Xa activity of a heparin-like substance produced by a heparin-like substance-producing animal cell; and
(F) a polynucleotide encoding a protein consisting of an amino acid sequence having an identity of 90% or higher to the amino acid sequence of SEQ ID NO: 30 or 44 and having a function to enhance the anticoagulant factor Xa activity of a heparin-like substance produced by a heparin-like substance-producing animal cell.
